(19) United States
(12) Patent Application Publication
(10) Pub. No.: US 2018/0075223 A1
(43) Pub. Date: Mar. 15, 2018

(54) POINT OF CARE TESTING (POCT) SYSTEM

(71) Applicant: Roche Diagnostics Operations, Inc., Indianapolis, IN (US)

(72) Inventors: Alejandro Morcillo Montejo, Lloret de Mar (ES); Dmytro Rud, Huenenberg See (CH)

(73) Assignee: Roche Diagnostics Operations, Inc., Indianapolis, IN (US)

(21) Appl. No.: 15/813,334

(22) Filed: Nov. 15, 2017

Related U.S. Application Data
(63) Continuation of application No. PCT/EP2016/060482, filed on May 10, 2016.

(30) Foreign Application Priority Data
May 26, 2015 (EP) 15169259.7

(51) Int. Cl. G06F 19/00

(52) U.S. Cl. CPC G06F 19/366; G06F 19/345; H04L 63/08; G06F 19/3418; G06F 19/327

(57) ABSTRACT

Disclosed are a system architecture and a method to enable workflow solutions via remote configuration services for the configuration management of point of care analyzers. According to particular embodiments, the disclosed system and method are configured to enable remote configuration services of point of care analyzers across different platforms.

### CROSS-REFERENCE TO RELATED APPLICATIONS

This application is a continuation of PCT/EP2016/060482, filed May 10, 2016, which is based on and claims priority to EP 15169259.7, filed May 26, 2015, which is hereby incorporated by reference.

### BACKGROUND

The present disclosure relates to a point of care testing (POCT) system and a method for configuration of a POCT system.

In vitro diagnostic testing has a major effect on clinical decisions, providing physicians with pivotal information. Particularly, there is great emphasis on providing quick and accurate test results in critical care settings.

One field of diagnostic testing is conducted with large analytical instruments in laboratories. These instruments are operated by operators that are educated to maintain and operate such instruments.

Another field of diagnostic testing is bedside testing or point of care testing (POCT). This type of diagnostic testing is performed mainly by nurses or medical staff primarily trained to operate the instruments available at the site of patient care, such as hospitals, emergency departments, intensive care units, primary care setting, medical centers, patient homes, a physician's office, a pharmacy or a site of an emergency.

Often, POCT needs to meet clinical and laboratory requirements for short turnaround times in critical care. Rapid determination of time-critical parameters (e.g. blood glucose, cardiac markers, blood gases, etc.) can accelerate decision making in the emergency room, intensive care units or even in the primary care setting.

Major benefits are obtained when the output of a POCT device is made available immediately. Results can be shared instantaneously with all members of the medical team, thereby enhancing communication through decreasing turnaround time.

POCT has become established worldwide and finds vital roles in public health. Potential operational benefits of point of care testing POCT include: faster decision making, reduced operating times, reduced postoperative care time, reduced emergency room time, reduced number of outpatient clinic visits, reduced number of hospital beds required and overall optimal use of professional time.

While there are many benefits of using POCT devices in terms of their convenience, establishing POCT is challenging. Some of the biggest challenges relate to engaging health care providers as testing personnel, all the while ensuring adherence to best laboratory practices and regulatory agency standards. Thus, POCT implementation requires a systematic approach that involves all stakeholders.

Implementation of configuration management workflows for point of care analyzer(s) poses further difficulties due to the high degree of complexity and lack of modularity and adaptability of existing system architectures for point of care testing POCT systems.

Furthermore, the fact that point of care analyzer(s) are by their nature often distributed throughout one or more medical units or facilities, further complicates management of point of care testing POCT systems.

### SUMMARY

According to the present disclosure, a point of care testing (POCT) method and system for analyzing biological samples are presented. The POCT system can comprise one or more point of care analyzer(s) for analyzing one or more patient sample(s). The one or more point of care analyzer(s) can be configured to receive the analyzer update command and to update at least one analyzer parameter according to the respective analyzer parameter update. The POCT system can also comprise a portable computing device and a hardware management server for storing system parameter(s) corresponding to the one or more point of care analyzer(s). The hardware management server can be configured to receive and process the configuration request in order to: update at least one system parameter according to the configuration request and transmit an analyzer update command comprising at least one analyzer parameter update to one or more point of care analyzer(s) according to the configuration request. The POCT system can also comprise a remote configuration server. The remote configuration server can be configured to: authenticate and authorize the portable computing device, receive a configuration request from the portable computing device, and transmit the configuration request to the hardware management server. The POCT system can also comprise a communication network configured to communicatively connect: the point of care analyzer(s) with the hardware management server, the portable computing device with the remote configuration server, and the remote configuration server with the hardware management server.

Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a simplified diagram of the disclosed point of care testing (POCT) system according to an embodiment of the present disclosure.

FIG. 2 illustrates a use case diagram of a disclosed method for configuration of a point of care testing (POCT) system according to an embodiment of the present disclosure.

FIG. 3 illustrates an illustrative diagram of the disclosed POCT system, including a hardware management server and a remote configuration server according to an embodiment of the present disclosure.

FIG. 4 illustrates a use case diagram of a disclosed method for configuration of a POCT system including a remote configuration server and a hardware management server according to another embodiment of the present disclosure.

FIG. 5A illustrates an illustrative diagram of the disclosed POCT system, the remote configuration server further including an authentication and authorization unit and an adapter unit according to an embodiment of the present disclosure.

FIG. 5B illustrates an illustrative diagram of the disclosed POCT system, communicatively connected to a Laboratory Information System (LIS) and/or Hospital Information system (HIS) according to another embodiment of the present disclosure.

FIG. 6 illustrates an illustrative diagram of the disclosed POCT system, the hardware management server including a first hardware management subserver communicatively connected to a first group of one or more point of care analyzer(s) and a second hardware management subserver communicatively connected to a second group of one or more point of care analyzer(s) according to an embodiment of the present disclosure.

FIG. 7 illustrates a use case diagram of a disclosed method for configuration of a POCT system including a remote configuration server and first and second hardware management servers according to another embodiment of the present disclosure.

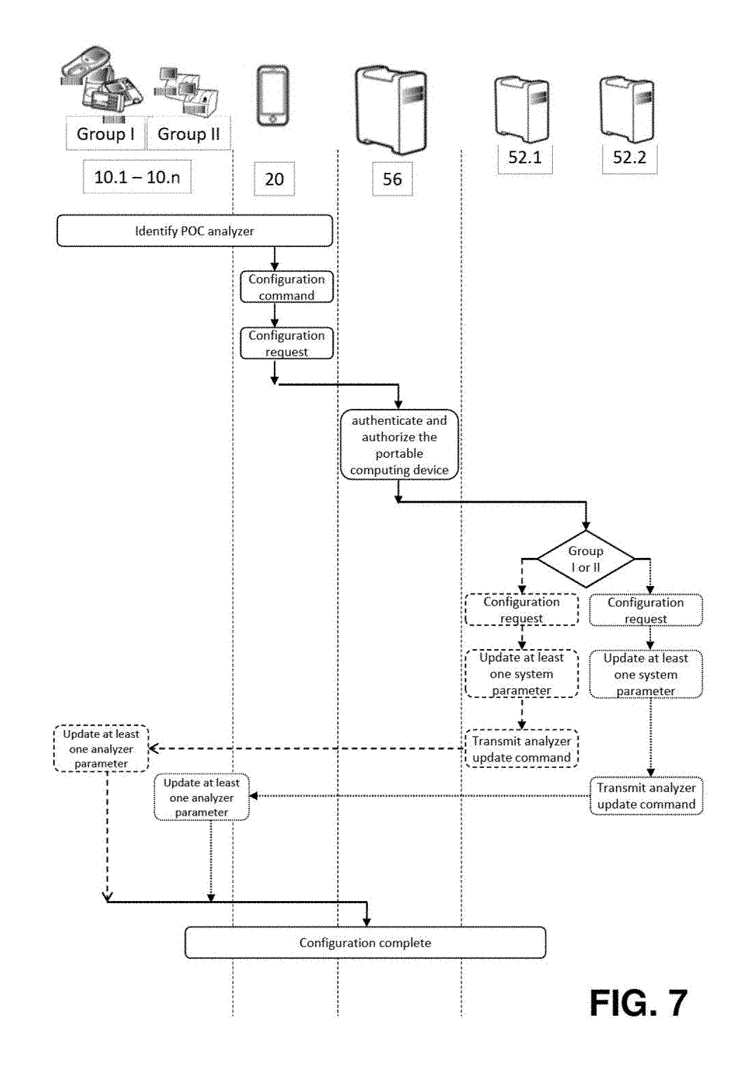

FIG. 8A illustrates an illustrative overview of the disclosed communication network according to an embodiment of the present disclosure.

FIG. 8B illustrates an illustrative overview of the disclosed communication network according to another embodiment of the present disclosure.

FIG. 8C illustrates an illustrative overview of the disclosed communication network according to another embodiment of the present disclosure.

FIG. 8D illustrates an illustrative overview of the disclosed communication network according to yet another embodiment of the present disclosure.

FIG. 9 illustrates a use case diagram of a disclosed method for configuration of a POCT system that includes relocation of a point of care analyzer according to an embodiment of the present disclosure.

FIGS. 10A-B illustrate screenshots of a portable computing device; illustrating method steps of a relocation of a point of care analyzer according to an embodiment of the present disclosure.

FIG. 11 illustrates a use case diagram a disclosed method for configuration of a POCT system, illustrating replacement of a first (defective) point of care analyzer with a second (replacement) point of care analyzer according to an embodiment of the present disclosure.

FIGS. 12A-D illustrate illustrative screenshots of a portable computing device; illustrating method steps of for replacement of a first (defective) point of care analyzer with a second (replacement) point of care analyzer according to an embodiment of the present disclosure.

FIG. 13 illustrates a use case diagram of the disclosed method for configuration of a POCT system, illustrating certification configuration corresponding to one or more operator(s) and one or more certification(s) according to an embodiment of the present disclosure.

FIGS. 14A-D illustrate illustrative screenshots of a portable computing device, illustrating method steps for certification configuration corresponding to one or more operator(s) and one or more certification(s) according to an embodiment of the present disclosure.

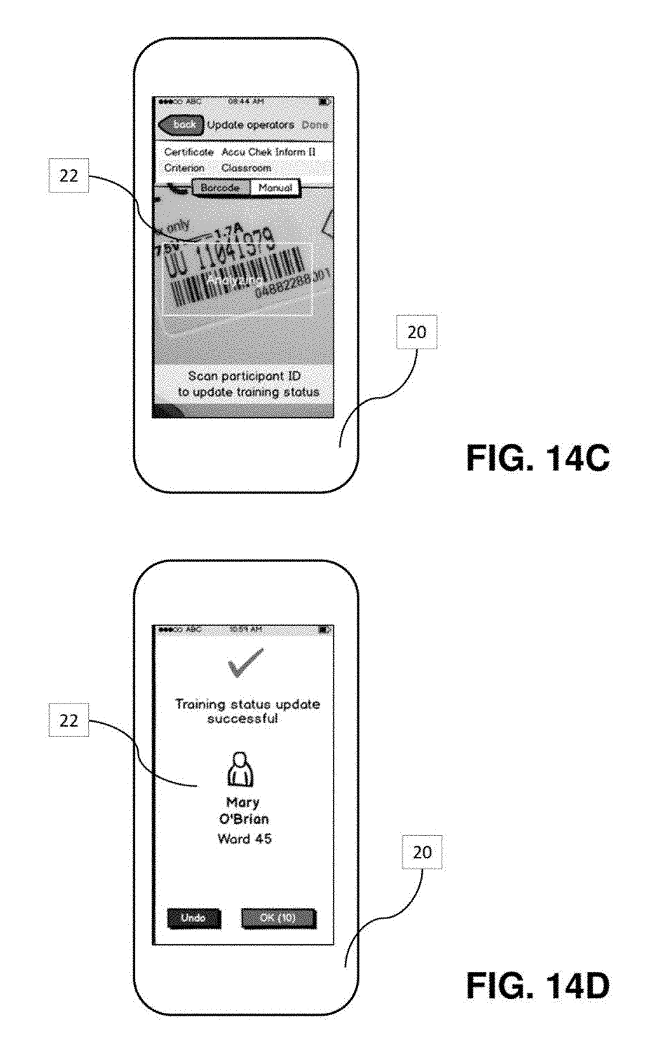

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

Disclosed are system architecture and a method to enable workflow solutions via remote configuration services for the configuration management of point of care analyzers.

In some embodiments, the disclosed system and method can be configured to enable remote configuration services of point of care analyzers across different platforms.

In some embodiments, the disclosed system and method can provide for convenient assisted workflows that enable efficient replacement of point of care analyzers and enable traceable relocation of point of care analyzers and efficient management of point of care analyzer operator certifications.

Certain terms will be used in this patent application, the formulation of which should not be interpreted to be limited by the specific term chosen, but as to relate to the general concept behind the specific term.

As used herein, the terms ‘comprises,’ ‘comprising,’ ‘includes,’ ‘including,’ ‘has,’ ‘having’ or any other variation thereof, are intended to cover a non-exclusive inclusion of features.

The terms ‘patient sample’ and ‘biological sample’ can refer to material(s) that may potentially contain an analyte of interest. The patient sample can be derived from any biological source, such as a physiological fluid, including blood, saliva, ocular lens fluid, cerebrospinal fluid, sweat, urine, stool, semen, milk, ascites fluid, mucous, synovial fluid, peritoneal fluid, amniotic fluid, tissue, cultured cells, or the like. The patient sample can be pretreated prior to use, such as preparing plasma from blood, diluting viscous fluids, lysis or the like. Methods of treatment can involve filtration, distillation, concentration, inactivation of interfering components, and the addition of reagents. A patient sample may be used directly as obtained from the source or used following a pretreatment to modify the character of the sample. In some embodiments, an initially solid or semi-solid biological material can be rendered liquid by dissolving or suspending it with a suitable liquid medium. In some embodiments, the sample can be suspected to contain a certain antigen or nucleic acid.

The term ‘analyzer’ as used herein can encompass any apparatus for obtaining a measurement value from a patient sample. For example, the analyzer can measure light absorption, fluorescence, electrical potential or other physical or chemical characteristics of a reaction or physical process to provide the measurement data. Often such patient samples are treated before analytical testing can be done. Blood sampled from a patient can be e.g. centrifuged to obtain serum or treated with anti-coagulants to obtain plasma.

Analytical testing by an analyzer can have the goal to determine the presence and/or concentration of an analyte in a patient sample. The term ‘analyte’ is a general term for substances for which information about presence and/or concentration is intended. Examples of analytes are e.g. glucose, coagulation parameters, endogenic proteins (e.g. proteins released from the heart muscle), metabolites, nucleic acids and so on. The term ‘patient health parameter’ as used herein can encompass any aspect of a patient's physiology that can be measurable or indicated by an analysis of a patient or patient sample for one or more analyte(s), for example blood pressure, blood oxygen level, body temperature, Urine specific gravity, or the like.

The term ‘analytical data’ as used herein can encompass any data that can be descriptive of a result of a measurement of one or more patient health parameter(s) performed by a point of care analyzer of the biological sample that has been analyzed. In case of a calibration the analytical data can include the calibration result, i.e. calibration data. In particular, the analytical data can include an identifier of the patient sample for which the analysis has been performed and data being descriptive of a result of the analysis, such as measurement data.

The term ‘workflow’ as used herein can encompass any task carried out by a human a machine, or a combination of a human and a machine that can include one or more steps, such as for maintenance or operation of a system or one of its system components.

The term ‘step of a workflow’ as used herein can encompass any activity belonging to a workflow.

The term ‘authentication data’ as used herein can encompass any kind of data suitable to identify an operator/user, such as a user name/user ID and/or password, a security token, a biometric identifier(s) or the like.

The term ‘authentication and authorization unit’ as used herein can encompass any hardware-, firmware- and/or software-based module operable to execute program logic for receiving and processing authentication data. Furthermore, the authentication and authorization unit can include any hardware-, firmware- and/or software-based module operable to execute program logic for determining, if the authenticated user/operator possesses the authorization to access a requested feature/data/resource/process/or the like.

The term ‘point of care (POC),’ point of care (POC) or ‘point of care POC environment’ as used herein can be defined to mean a location on or near a site of patient care where medical or medically related services such as medical testing and/or treatment are provided, including but not limited to hospitals, emergency departments, intensive care units, primary care setting, medical centers, patient homes, a physician's office, a pharmacy or a site of an emergency.

The term ‘point of care testing (POCT)’ as used herein can encompass analysis of one or more patient(s) or patient sample(s) for one or more patient health parameters in a point of care (POC) environment. Point of care testing (POCT) can be often accomplished through the use of transportable, portable, and handheld instruments, but small bench-top analyzers or fixed equipment can also be used when a handheld device is not available—the goal being to collect the patient health parameter and obtain analytical data in a (relatively) short period of time at or (relatively) near the location of the patient.

The term ‘point of care analyzer’ as used herein can encompass any analyzer used in a point of care (POC) environment, such as (but not limited to) blood glucose testing, coagulation testing, blood gas and electrolytes analysis, urinalysis, cardiac markers analysis, hemoglobin diagnostics, infectious disease testing, cholesterol screening or nucleic acid testing. Results may be viewed directly on the point of care analyzer(s) or may be sent to the point of care testing (POCT) system and displayed in a Laboratory Information System with central lab results, or alongside other results in a Hospital Information System.

The term ‘portable computing device’ as used herein can encompass any electronic appliance that can be moved from one location to another without the need of using a tool to sever a connection of the appliance with another appliance, in particular, any handheld battery powered mobile appliance, including but not limited to a cellular telephone, a satellite telephone, a pager, a personal digital assistant (PDA), a smartphone, a navigation device, a smartbook or reader, a combination of the aforementioned devices, a tablet computer or a laptop computer.

The term ‘communication network’ as used herein can encompass any type of wired or wireless network, including but not limited to a WIFI, GSM, UMTS or other wireless digital network or a wired network, such as Ethernet or the like. For example, the communication network may include a combination of wired and wireless networks.

The term ‘server’ as used herein can encompass any physical machine or virtual machine having a physical or virtual processor, capable of accepting requests from and giving responses accordingly. It can be clear to a person of ordinary skill in the art of computer programming that the term machine may refer to a physical hardware itself, or to a virtual machine such as a JAVA Virtual Machine (JVM), or even to separate virtual machines running different Operating Systems on the same physical machine and sharing that machine's computing resources. Servers can run on any computer including dedicated computers, which individually can also be often referred to as ‘the server’ or shared resources such as virtual servers. In many cases, a computer can provide several services and have several servers running. Therefore, the term server can encompass any computerized device that shares a resource with one or more client processes.

The term ‘subserver’ as used herein can encompass any type of hardware-, firmware- and/or software-based module which may be functionally and/or structurally part of a server. Therefore any server which is itself part of a server may be referred to as a subserver. Nevertheless any server as defined above which can be part of a system may be referred to as a subserver.

The term ‘hardware management server’ as used herein can encompass any type of server which can be configured to manage and to provide connectivity to one or more point of care analyzers. A hardware management server can also be referred to as a point of care (POC) data management system or generically a middleware. In particular, the term ‘hardware management server’ as used herein encompasses IT solutions for the POC environment, such as the cobas IT 1000 application from Roche Diagnostics (Mannheim, Germany). According to certain embodiments, the hardware management server can provide not only connectivity for point of care analyzers but also management of test results, operators, quality controls, and analyzers.

The term ‘remote configuration server’ as used herein can encompass any type of server configured to provide remotely accessible configuration services, in particular configuration services accessible through a portable computing device.

The term ‘server interface’ as used herein can encompass any hardware-, firmware- and/or software-based module operable to execute program logic to allow communication with an external entity (such as a server or another interface). An interface can be a boundary across which two separate components of a system exchange data/information. The exchange can be between software, computer hardware, peripheral devices, humans and combinations of these.

The term ‘expose’ as used herein—with reference to a server exposing an interface—can refer to a server enabling a separate entity (e.g. another server/subserver or another interface) access to certain of its features, data, functionality via the interface.

The term ‘adapter unit’ as used herein can encompass any type of server/subserver or any hardware-, firmware- and/or software-based module configured to connect multiple software and/or hardware entities. The functionalities and configuration of the adapter according to the disclosed system/method will be described in corresponding paragraph(s) below.

The term ‘user interface’ as used herein can encompass any suitable piece of software and/or hardware for interactions between an operator and a machine, including but not limited to a graphical user interface for receiving as input a command from an operator and also to provide feedback and convey information thereto. Also, a system/device may expose several user interfaces to serve different kinds of users/operators.

In the field of bedside testing or point of care testing (POCT), the testing is done on patients typically by nurses, medical staff or doctors but also pharmacists who are collectively can be referred to as ‘operator(s)’ herein. However, anyone who possesses the required certification may be an operator. A point of care coordinator (POCC) may be at the same time an operator of point of care analyzer(s) and also an operator of point of care analyzer(s) may be at the same time a POCC and thus user of portable computing device(s).

The term ‘certification’ as used herein can encompass any form of confirmation of certain characteristics (such as training and/or examination and/or educational background and/or accreditation) of an operator. In particular, a certification as disclosed herein may not be restricted to embodiments which are formally titled “certification” or physical embodiments (such as a printed certification) having a related title. According to embodiments of the disclosed system/method, certification(s) can be provided by an entry of an operator on list(s) of certified operators allowed to perform a job/task/workflow or step of a workflow using one or more of the point of care analyzers. The certification(s) according to embodiments of the disclosed system/method may be permanent and/or time-restricted certifications, meaning that the certification corresponding to an operator may become invalid after a certain period of time. After a certification becomes invalid and the respective operator needs to become certified again (by taking a training and/or passing a (re)examination), otherwise that operator may no longer use the respective point of care analyzer(s) or certain features/functions thereof.

The term ‘system certification’ as used herein can encompass a certification stored on a server. According to embodiments, the system certification relates to all point of care analyzers of the POCT system. According to further embodiments, the system certification can relate to one or more point of care analyzers of a certain type, class, namely point of care analyzers having at least one common characteristic. Examples of common characteristics of one or more point of care analyzers are: point of care analyzers being capable of performing the same or similar analyses of patient(s) or patient sample(s); point of care analyzers requiring the same or similar operator training/examination/certification; point of care analyzers from one manufacturer; point of care analyzers at the same healthcare facility, etc.

The term ‘analyzer certification’ as used herein can encompass any certification stored on a point of care analyzer. According to some embodiments, the analyzer certification can store any kind of representation of the operators (such as a list of operator identifiers) authorized to perform at least one job/task/workflow or step of a workflow using that particular point of care analyzer, in particular operators authorized to analyze one or more patient sample(s) using that particular point of care analyzer. According to certain embodiments, each analyzer certification can be specific to one particular point of care analyzer. According to further embodiment(s), each analyzer certification can be specific to one or more point of care analyzers of a certain type, class. According to even further embodiment(s), the analyzer certification(s) may be identical to the system certification(s), but stored locally on the point of care analyzer(s).

Since point of care testing (POCT) performed near the patient can lead directly to diagnostic and therapeutic decisions, a POCT system advantageously can meet multiple requirements (similar to requirements in laboratory testing, but often within shorter times). Examples of such requirements include:
Provide accurate and timely analyses and associate them with the correct patient;
Ensure that operators are competent/certified for the use of the POCT system;
Ensure proper operation, availability and configuration of the analyzers;
Provide reports that are useful to the clinician treating the patient; and
Document testing and Quality Control (QC) for audit purposes.

POCT can be performed using various point of care analyzers such as (but not limited to) analyzers for glucose, coagulation, blood gas, urinalysis, cardiac and molecular testing. Results may be viewed directly on the point of care analyzer(s) or may be sent to the POCT system and displayed in a Laboratory Information System with central lab results, or alongside other results in a Hospital Information System.

Point of care analyzers are commonly managed by a server, and in particular, a hardware management server, also referred to as point of care (POC) data management systems. Such a server can provide connectivity for point of care analyzers and management of test results, operators, quality controls, and analyzers.

Management of POCT can be challenging—there can be dozens of sites, hundreds of POCT devices/kits, and thousands of operators to manage to assure quality of testing. One challenge in developing a strategy to manage POCT usually can involve building a competent interdisciplinary POC management team including the laboratory, physicians, and nurses. The POC team can usually hold the responsibility for determining the test menu, selecting technologies, establishing policies and procedures, ensuring training and regulatory compliance, and providing advisory assistance to the end operators of POC technologies. After establishing a POC team, a management structure can often be built that is responsible to implement new initiatives and to perform corrective action where necessary. The point of care analyzers of a POCT system can generally be managed by one or more Point of care coordinator(s) (POCC). The POCC can be responsible for ensuring that all analyzers are up and running; that all operators are able to use the analyzer(s); know where the analyzers and operators are; and make sure the system is compliant with the regulatory requirements.

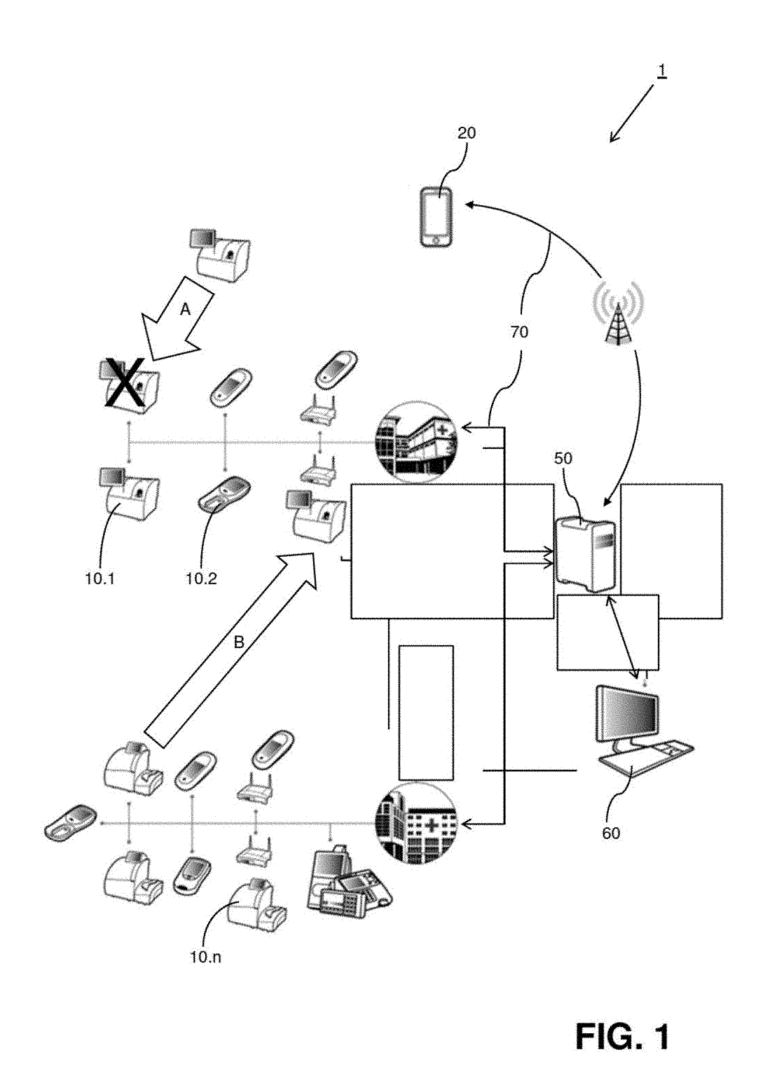

As illustrated on FIG. 1, for example, the point of care testing (POCT) system 1 as disclosed herein can include one or more point of care analyzer(s) 10.1-10.n, a portable computing device 20 and a server 50 communicatively connected by a communication network 70. In particular, the communication network 70 can be configured to communicatively connect the one or more point of care analyzer(s) 10.1-10.n and the portable computing device 20 with the server 50.

The point of care analyzer(s) 10.1-10.n can be provided and configured for analyzing one or more patient sample(s) in order to measure one or more patient health parameter(s). According to some embodiments, point of care analyzer(s) 10.1-10.n can include transportable, portable, and handheld instruments, along with small bench analyzers or fixed equipment as well, such as (but not limited to) blood glucose testing, coagulation testing, blood gas and electrolytes analysis, urinalysis, cardiac markers analysis, hemoglobin diagnostics, infectious disease testing, cholesterol screening or nucleic acid testing (NAT). Several functional and/or operational aspects of the point of care analyzer(s) 10.1-10.n can be configurable/customizable using one or more analyzer parameter(s).

In order to identify a particular point of care analyzer(s) 10.1-10.n, each can be provided with an analyzer identifier, in particular, in form of an identifier tag, such as a barcode and/or an RFID tag but can be a serial number as well.

The server 50 can be provided and configured for storing system parameter(s) corresponding to the one or more point of care analyzer(s) 10.1-10.n. Some of these system parameters can be common for more than one point of care analyzers while some system parameters can be specific to one single individual point of care analyzer 10.1-10.n.

The at least one system parameter and/or analyzer parameter can include (but are not limited to) one or more of the following:
Analyzer specific parameters:
   Formatting setting(s)
   Language setting(s),
   Date/Time format setting(s)
   Shutdown/Sleep/Hibernate/Logout timeout
   Connection configuration (e.g. wlan authentication data)
   Analyzer Status: in use, backup, etc.
Security parameters
   Authentication mechanism
   Login mechanism (only user ID, user ID and Pwd or Barcode scanning)
Patient identification/mapping parameters
   Patient identification mechanism
   Patient ID mapping
Measurement parameters
   Default measurement unit(s)
   Workflow definitions) (e.g. force having comments on results or set them optional)
   Ranges (such as reference ranges)
Quality Control QC parameters
   QC Lockout
   Lot verification
   QC result display
Location specific parameters
   Physical location: Healthcare Facility, Building, Floor, Unit, Room
   Logical location: Emergency room, intensive care units, primary care setting, medical center, patient home, a physician's office, a pharmacy or a site of an emergency
   Location-specific authentication and authorization data.

In certain embodiments of the disclosed system/method, the server 50 can be configured to: retrieve analytical data from the one or more point of care analyzer(s) 10.1-10.n such as data representing the measurement of patient health parameter(s) and update program data of the one or more point of care analyzer(s) 10.1-10.n such as a software update.

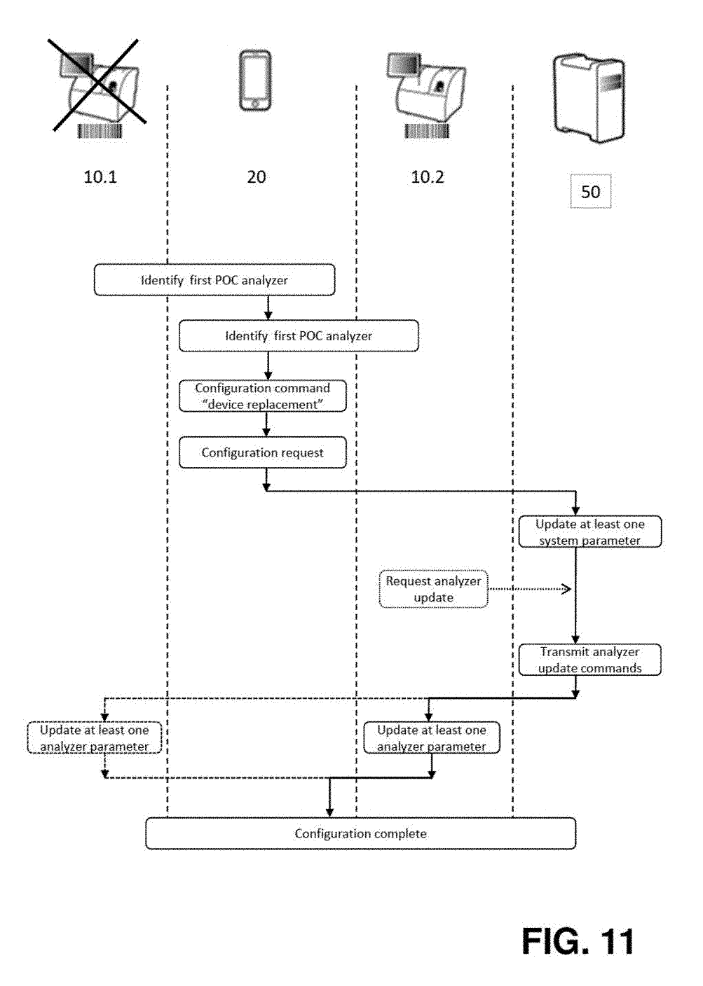

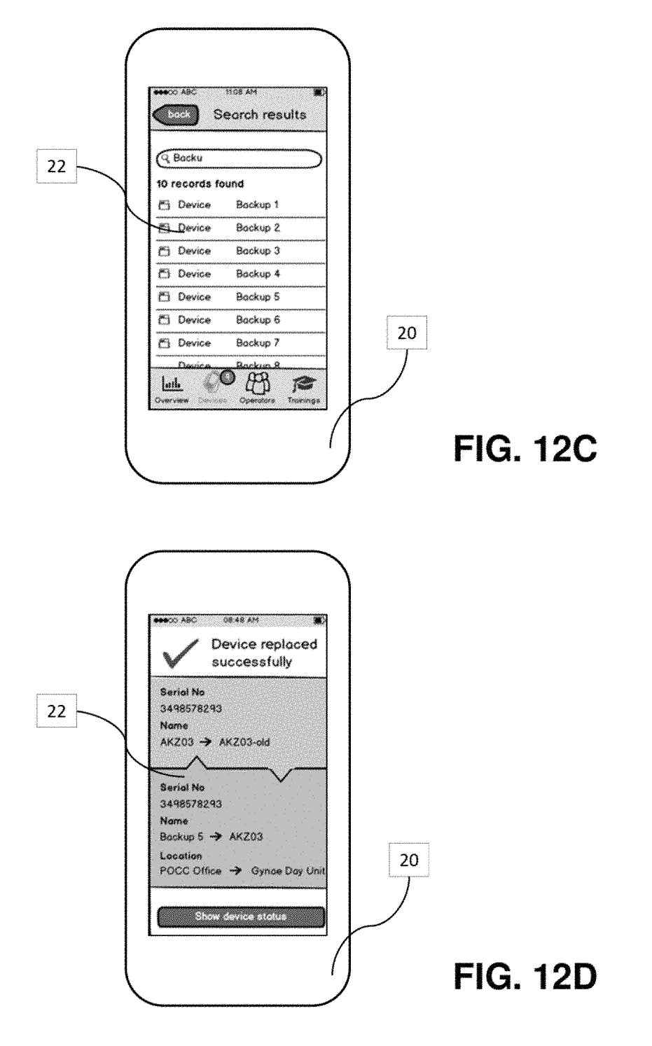

The block arrows A and B of FIG. 1 illustrate an analyzer replacement respectively an analyzer relocation workflow as described with reference to the use case diagrams of FIGS. 9 and 11.

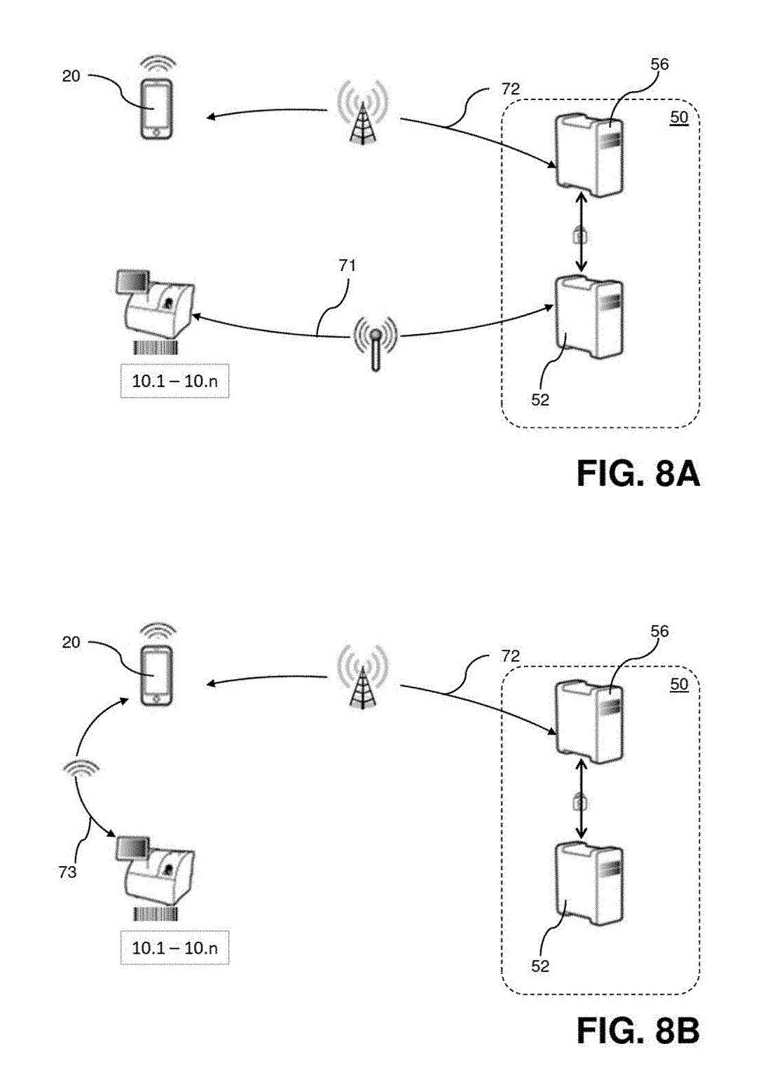

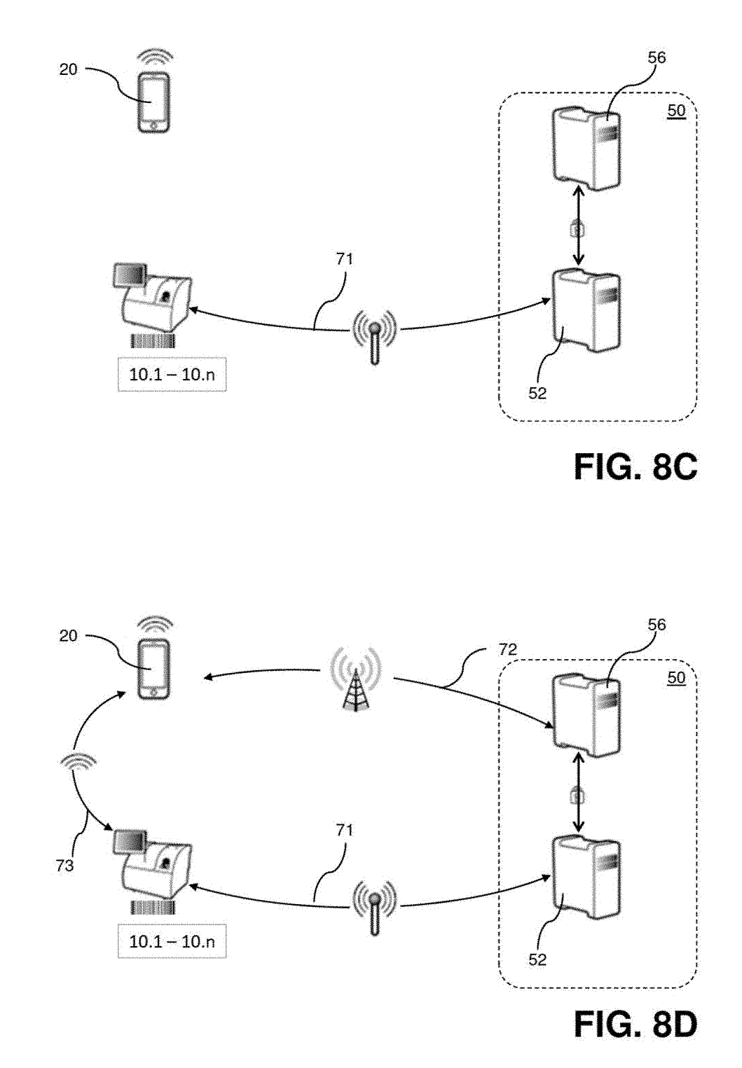

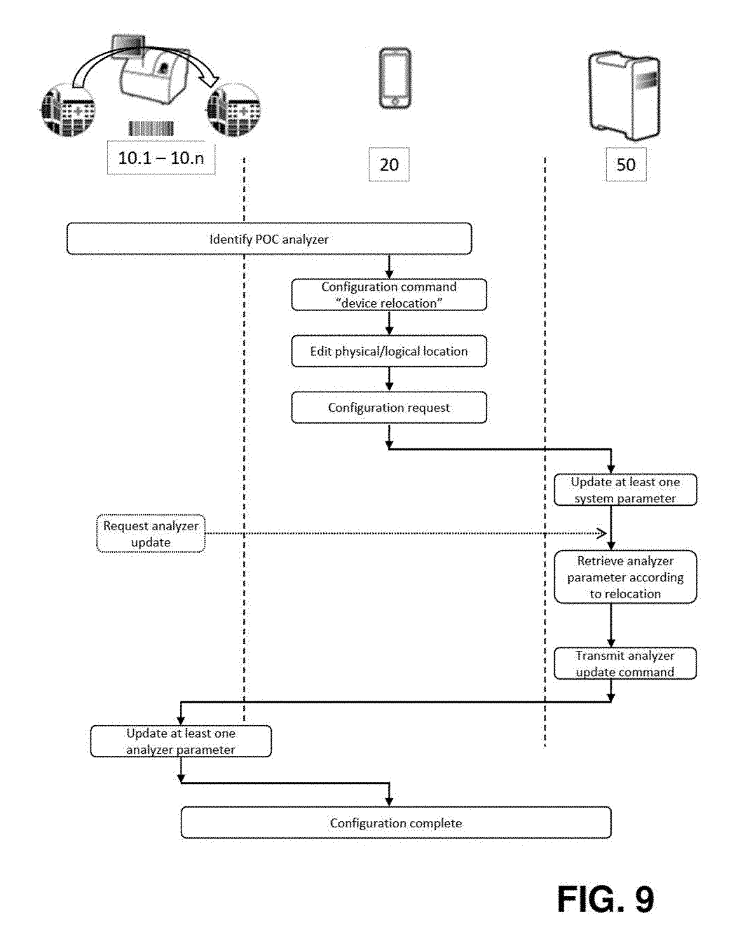

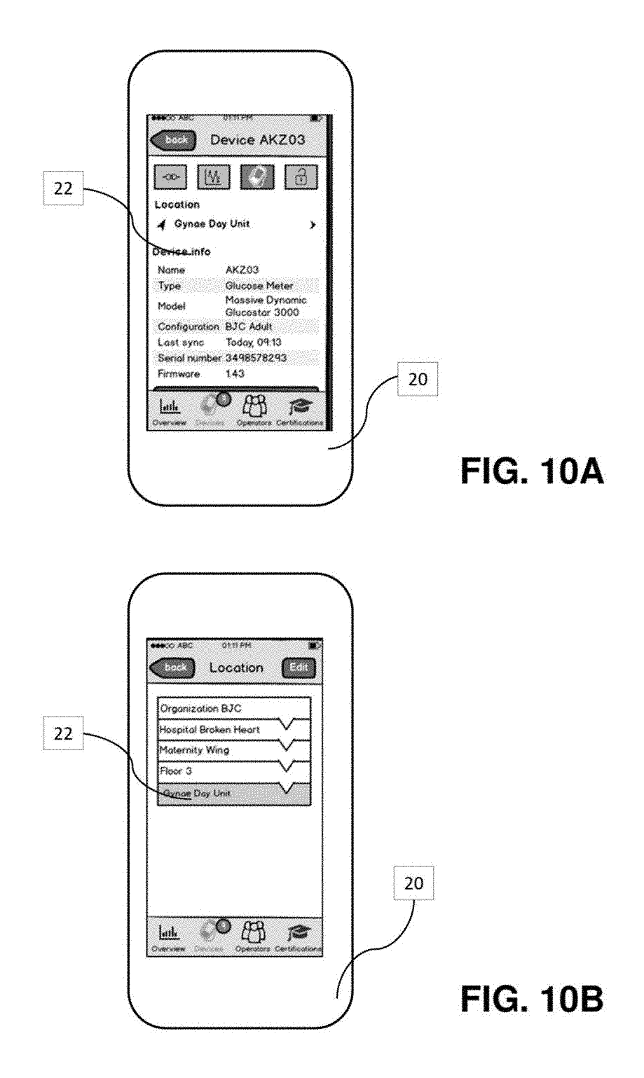

As illustratively shown on FIG. 1, the communication network 70 can be laid out/configured to communicatively connect the one or more point of care analyzer(s) 10.1-10.n and the portable computing device 20 with the server 50, FIGS. 8A to 8D illustrate different embodiments thereof.

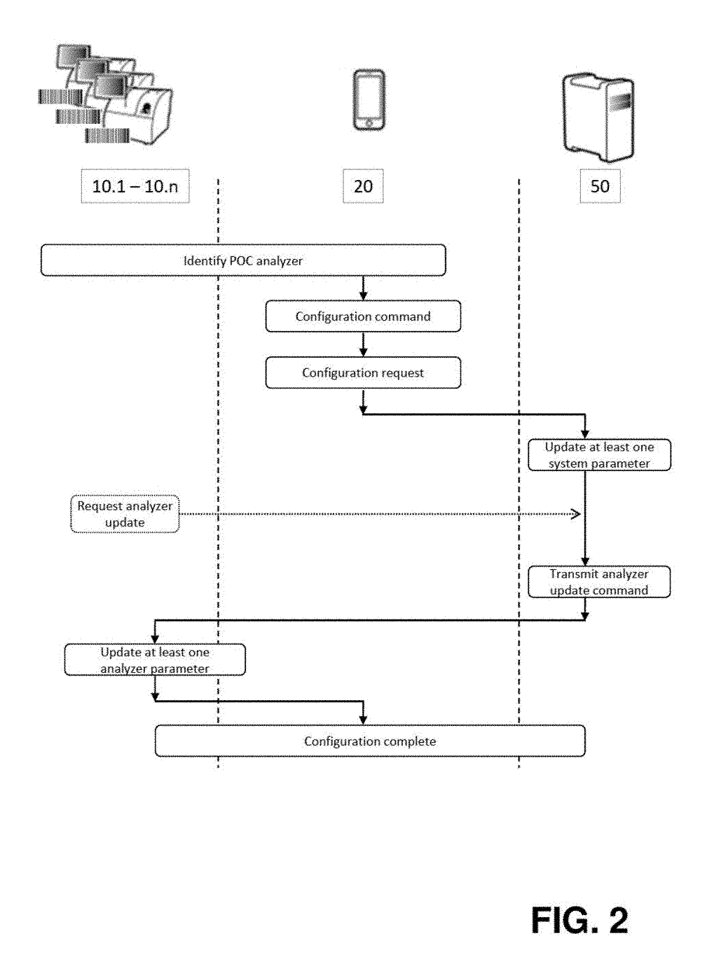

FIG. 2 shows a use case diagram of an embodiment of the disclosed method for configuration of a POCT system 1. The symbols on the top of the diagram illustrate the actors of the workflow—the point of care analyzers 10.1-10.n, the portable computing device 20 and the server 50, while the steps of the workflow are shown below, illustrating the “involvement” of the actors in the respective workflow step(s). Workflow steps of particular embodiment(s) only are illustrated with dotted lines.

In a step, the portable computing device 20 can identify the point of care analyzer(s) 10.1-10.n on basis of the respective analyzer identifier. Corresponding to the particular analyzer identifier, the portable computing device 20 can include an identifier reader such as a barcode reader and/or an RFID reader to read the identifier tag and/or a user interface for inputting a serial number of the one or more point of care analyzer(s) 10.1-10.n. Alternatively, an imaging device (such as a camera) of the portable computing device 20 can be configured to identify the point of care analyzer(s) 10.1-10.n. It can be noted that the portable computing device 20 can be configured such that it can identify more than one point of care analyzer(s) 10.1-10.n at the same time or successively and thus can initiate a reconfiguration of more than one point of care analyzers 10.1-10.n at the same time. The more than one point of care analyzers 10.1-10.n identified can be referred to as the identified point of care analyzer(s) 10.1-10.n.

In order to initiate/start a configuration workflow (process), a configuration command can be given (by an operator). Therefor the portable computing device 20 can be provided with a user interface 22 configured to receive the configuration command. According to some embodiments of the user interface 22, the configuration command may be a push of a button (physical or screen button) a voice command, a selection in a menu, and the like. The configuration command may be any form of input from an operator to initiate a configuration workflow (process).

Triggered by the configuration command, the portable computing device 20 can generate a configuration request including the analyzer identifier of the identified point of care analyzer(s) 10.1-10.n. After generating it (optionally after confirmation from the operator), the portable computing device 20 can transmit the configuration request to the server 50. Therefore, the configuration request can be described as a translation of the configuration command from the operator into a request signal to the server 50.

The server 50 can be configured to receive the configuration request, the transmission thereof being performed using the communication network 70. After receiving it, the server 50 can update at least one system parameter corresponding to the identified point of care analyzer(s) 10.1-10.n and transmit an analyzer update command including at least one analyzer parameter update to the identified point of care analyzer(s) 10.1-10.n.

The above steps by the server 50 will now be described in greater detail. On one hand, the server 50 can be configured to update at least one system parameter corresponding to the identified point of care analyzer(s) 10.1-10.n. The server 50 can retrieve (look up) the corresponding system parameter(s) as the configuration request generated by the portable computing device 20 can include the analyzer identifier(s). Depending on the configuration command/configuration request (as it will be described in following paragraphs of this specification), the server 50 can update the appropriate system parameter(s).

In order to ensure that the configuration command (as a complete workflow) is implemented not only on the server 50 but across the POCT system 1, the server 50 can be configured to transmit an analyzer update command(s) including at least one analyzer parameter update to the identified point of care analyzer(s) 10.1-10.n which in turn can be configured to receive the analyzer update command and to update at least one analyzer parameter according to the corresponding analyzer parameter update. According to embodiment(s) of the disclosed system/method and according to the particular point of care analyzer(s) 10.1-10.n, the updating of an analyzer parameter may become effective immediately, upon the next start-up/restart/docking, etc. of the analyzer.

In other words, the configuration command given via the user interface 22 of the portable computing device 20 can initiate a system-wide configuration workflow. Within the configuration workflow, the portable computing device 20, the server 50 and the point of care analyzers 10.1-10.n can collaborate (via the communication network) to ensure that required parameters (system parameters and analyzer parameters alike) are updated across the POCT system 1. This approach can have the advantage that a single configuration command can be given from a remote location (remote meaning a location different from the server) in order to (re)configure the POCT system 1—including any number of point of care analyzers, the POCT system 1 being configured such as to implement the configuration command without the need for additional actions/steps by the operator. Thus both operator effort and probability of operator error can be greatly reduced, as there is no need for the operator to individually update system parameters on the server 50, identify the corresponding analyzer parameters and then update the respective point of care analyzers 10.1-10.n. These steps can be taken over by the disclosed system/method. Therefore, embodiments of the disclosed system/method can be particularly advantageous over existing solutions, which commonly perform only the update of the system parameters on a server, wherein the operator is responsible to perform the update of the analyzer parameters of the respective point of care analyzer(s).

According to embodiments of the disclosed system/method, the server 50 transmitting an analyzer update command, the point of care analyzer(s) 10.1-10.n receiving the analyzer update command, and the point of care analyzer(s) 10.1-10.n updating of at least one analyzer parameter can be initiated by one or more point of care analyzer(s) 10.1-10.n requesting an analyzer update. Correspondingly, the one or more point of care analyzer(s) 10.1-10.n can be configured to request an analyzer update on occurrence(s) of certain event(s) and/or at regular intervals. According to embodiments of the disclosed system/method, such event(s) can include (but are not limited to) a login by an operator; a startup/shutdown/docking/undocking (into a docking station) of the point of care analyzer 10.1-10.n. According to embodiments of the disclosed system/method, the regular intervals (when the one or more point of care analyzer(s) 10.1-10.n can be configured to request an analyzer update) can be chosen to coincide with work shift changes of the operators or with time intervals when—based on historical data—the point of care analyzers are least expected to be used. Thus the regular intervals can be configured such as to minimize unavailability (or predicted unavailability) of the point of care analyzer(s) 10.1-10.n.

To summarize the analyzer update(s)—according to embodiment(s) of the disclosed system/method—the analyzer update(s) may be implemented using “server push” technology (that is server initiated) and/or a “client pull” technology (client—in this case point of care analyzer initiated). The server push implementation of the analyzer update can be advantageous for point of care analyzers 10.1-10.n which are continuously communicating with the server 50, while the client pull implementation of the analyzer update can be advantageous for point of care analyzers 10.1-10.n which communicate with the server 50 only on an event basis, such as periodically and/or upon a login by an operator and/or upon startup and/or upon shutdown of the respective point of care analyzer 10.1-10.n.

Embodiments of the disclosed system/method can be particularly advantageous as they can allow a decentralized control of point of care analyzers by a portable computing device. The disclosed solution can allow communication with hardware management software (such as the COBAS IT 1000 of Roche Diagnostics) through a portable computing device, making it possible to (re)configure point of care analyzer(s) and at the same time also initiate the corresponding server update(s). This combined update of both system parameter(s) and analyzer parameter(s) can ensure consistent (re)configuration of the POCT system within a single workflow. The exposure by the POCT system of such workflows for the (re)configuration of point of care analyzers remotely by a portable computing device are collectively referred to as ‘remote configuration services’.

Further embodiments of the disclosed POCT system can be configured to enable efficient workflow solution(s) for the configuration management of point of care analyzer(s), which system architecture can be optimized such that existing hardware management server(s)/systems need little to no modifications. These further embodiments can address the difficulties in the implementation of configuration management workflows for point of care analyzer(s) due to the high degree of complexity and lack of modularity and adaptability of existing system architectures for POCT systems. In particular, embodiments herein disclosed can be configured to enable remote configuration services of point of care analyzers from different types, vendors which are connected to different standard and/or proprietary hardware management systems, by handling authentication and authorization data with respect to configuration management across different platforms.

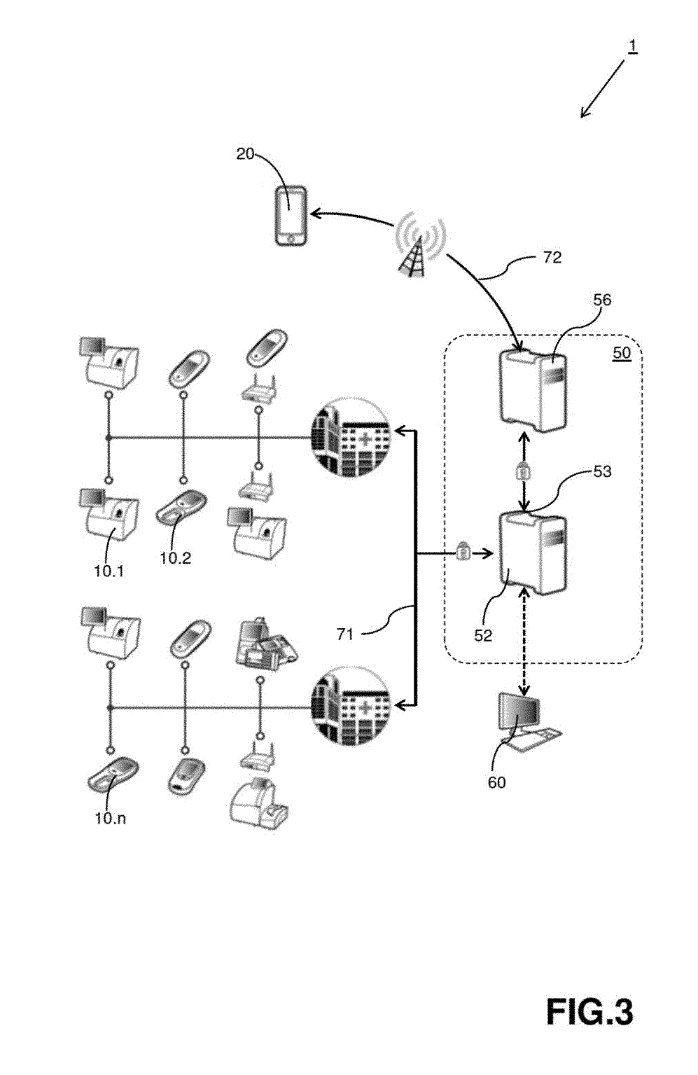

As illustrated on FIG. 3, according to further embodiments of the disclosed POCT system, the functionalities of the server 50 can be distributed between a hardware management server 52 and the remote configuration server 56. It can be noted that according to various embodiments, the hardware management server 52 and a remote configuration server 56 may or may not be one and the same hardware-, firmware- and/or software unit.

The hardware management server 52 can be provided and configured for storing the system parameter(s) (see definition above) corresponding to the point of care analyzer(s) 10.1-10.n. Also, it is the hardware management server 52 that can be communicatively connected to the point of care analyzer(s) 10.1-10.n, via the communication network 70.

As shown on FIG. 3, a management console 60 may be provided to access the hardware management server 52.

According to embodiments of the disclosed system/method, the hardware management server 52 can be configured to expose a hardware management server interface 53 for communication with the remote configuration server 56.

The disclosed embodiments of the remote configuration server 56 can allow consumer systems—that is the hardware management server(s) 52 to resolve what are the resources and actions exposed. This can support easy development of interfaces which have to connect to the remote configuration server 56.

While not shown on the figures, the hardware management server 52 can be further configured to perform one or more of the following: retrieve analytical data from the point of care analyzer(s) 10.1-10.n, update program data of the point of care analyzer(s) 10.1-10.n, and transfer of authentication data update(s) and/or certification update data of the point of care analyzer(s) 10.1-10.n.

According to embodiments of the disclosed system/method, the communication network 70 can include a point of care (POC) communication network area 71 for communicatively connecting the point of care analyzer(s) 10.1-10.n with the hardware management server 52 and a remote configuration network area 72 for communicatively connecting the portable computing device 20 with the remote configuration server 56.

Particulars of the communication network 70 are described with reference to FIGS. 8A through 8D.

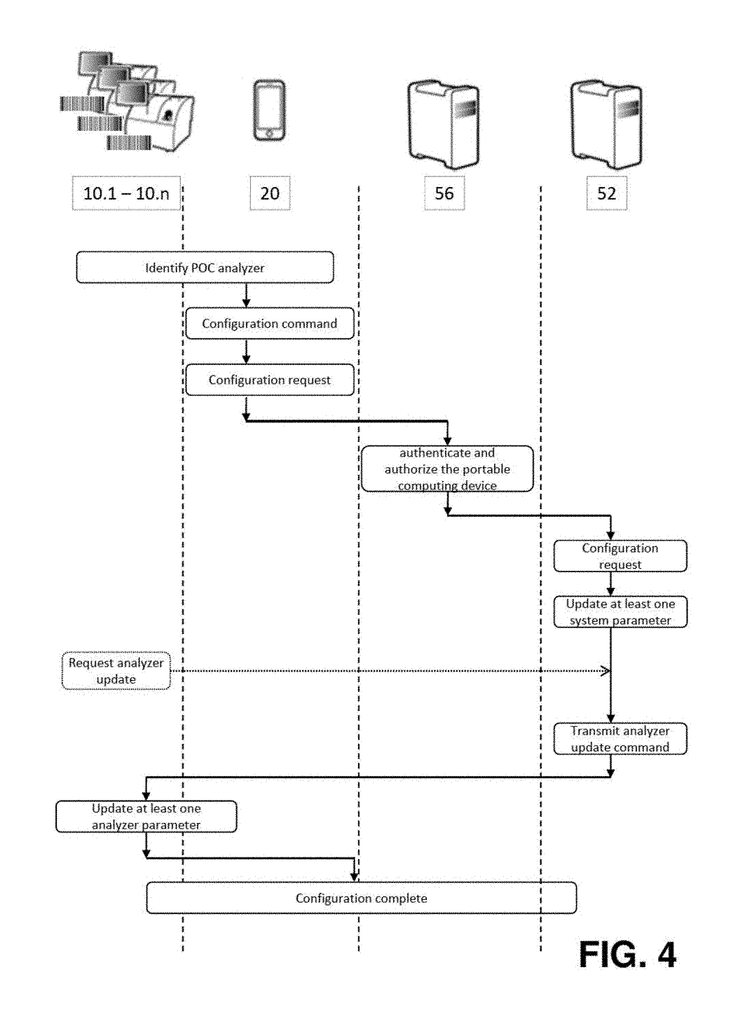

FIG. 4 shows a use case diagram of an embodiment of a disclosed method for configuration of a POCT system 1 including a remote configuration server 56 and a hardware management server 52.

In a step, the portable computing device 20 can identify the point of care analyzer(s) 10.1-10.n on basis of the respective analyzer identifier. Thereafter, in order to initiate/start a configuration workflow (process), a configuration command can be given (by an operator) via a user interface 22 of the portable computing device 20.

Triggered by the configuration command, the portable computing device 20 can generate a configuration request including the analyzer identifier of the identified point of care analyzer(s) 10.1-10.n. After generating it (optionally after confirmation from the operator), the portable computing device 20 can transmit the configuration request to the remote configuration server 56.

Being communicatively connected by the communication network 70 to the portable computing device 20, the remote configuration server 56 can authenticate and authorize the portable computing device 20. The authentication of the portable computing device 20 can be based on authentication data, such as a user name/user ID and/or password, a security token, a biometric identifier(s) or the like to authenticate the portable computing device 20 and/or an operator thereof. After authentication, the remote configuration server 56 can determine if the authenticated portable computing device 20, respectively a user thereof, possesses the authorization to access a requested feature/data/resource/process/or the like, in particular whether the authenticated portable computing device 20 is allowed to carry out the workflow initiated by the configuration request.

Since the task of authenticating and authorizing the portable computing device 20 is handled by the remote configuration server 56, the management of the remote configuration services (via the portable computing device 20) can be handled by the remote configuration server 56 and may be transparent to the hardware management server 52 and the point of care analyzer(s) 10.1-10.n. Such embodiments of the disclosed system/method can be advantageous as the use of a dedicated remote configuration server 56 for connecting the portable computing device(s) 20 can allow implementing remote configuration of existing POCT systems. Thus, in particular embodiments, remote configuration of existing POCT systems may be implemented fully agnostic to the hardware management server 52 and the point of care analyzer(s) 10.1-10.n. In other words, there may be no need to change the hardware management server 52 and the point of care analyzer(s) 10.1-10.n. On the other hand, the underlying hardware management server 52 can be exchanged, substituted or scaled without affecting the remote configuration server 56 and thus the remote configuration of the POCT system 1 via portable computing device(s) 20.

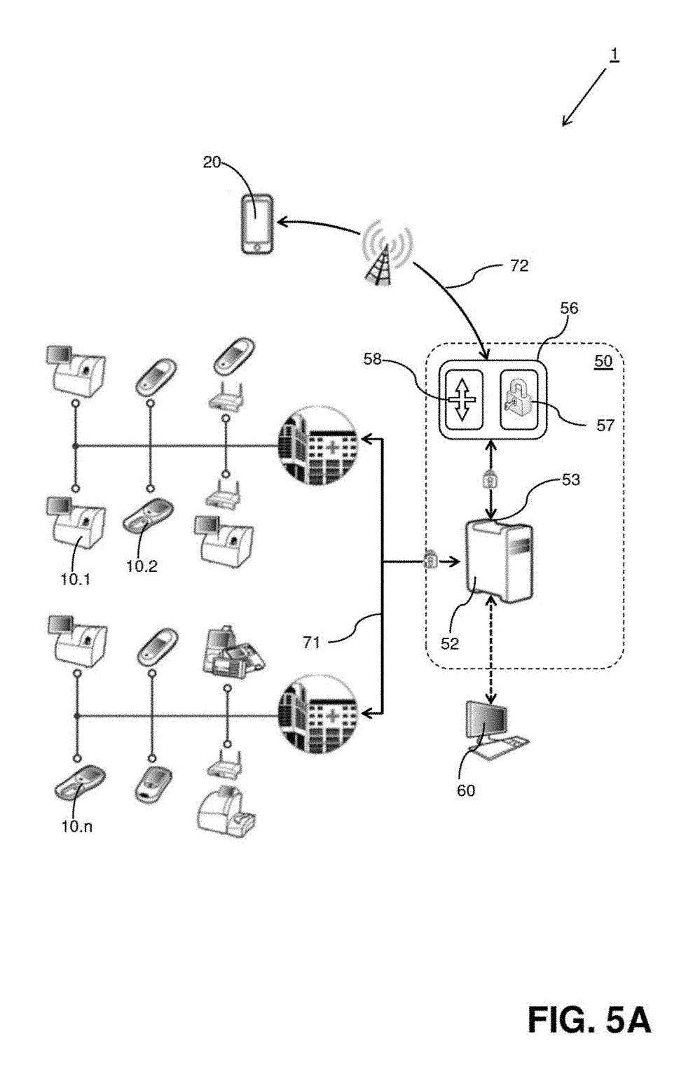

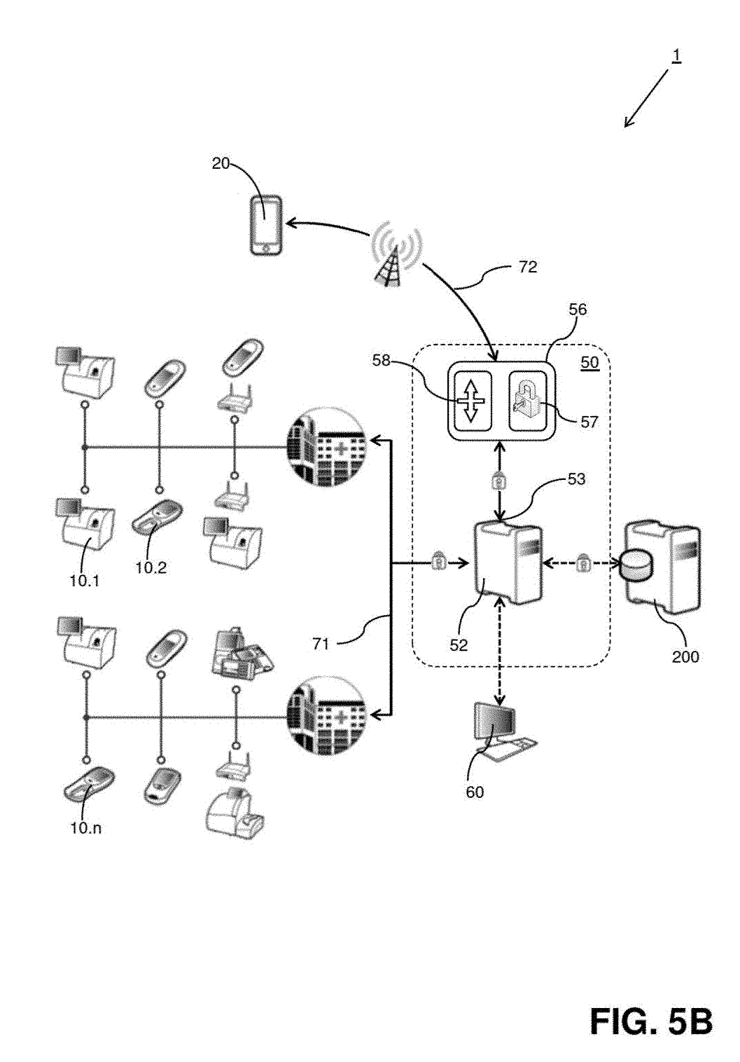

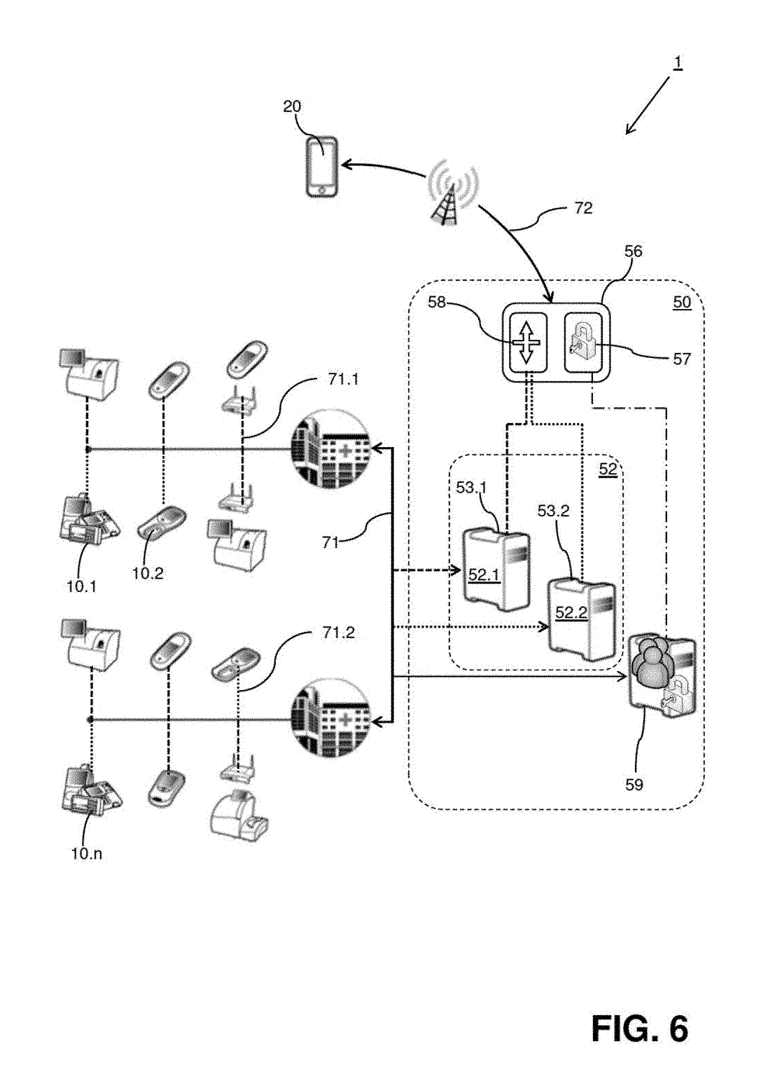

After successful authentication and authorization of the portable computing device 20, the remote configuration server 56 can receive/accept the configuration request from the portable computing device 20. Thereafter—being communicatively connected by the communication network 70 to the hardware management server 52—the remote configuration server 56 can be further configured to forward the configuration request(s) to the hardware management server 52. As it will be described in relation to FIGS. 5A, 5B and 6, according to particular embodiments, the remote configuration server 56 can be configured to adapt the configuration request received from the portable computing device 20 before transmitting the configuration request to the hardware management server 52, in accordance with the particular hardware management server 52.

Therefore, according to embodiments of the disclosed system/method, the remote configuration server 56 can take over both the communication with and also the authentication/authorization of the portable computing device(s) 20. Thus, it is the remote configuration server 56 which can expose the remote configuration services to the portable computing device(s) 20, ensuring itself compatibility with the particular hardware management server 52.

The hardware management server 52 can then receive the configuration request from the remote configuration server 56, the transmission thereof being performed using the communication network 70. After receiving it, the hardware management server 52 can update at least one system parameter corresponding to the identified point of care analyzer(s) 10.1-10.n. In order to ensure that the configuration command (as a complete workflow) is implemented not only on the hardware management server 52 but across the POCT system 1, the hardware management server 52 can be configured to transmit an analyzer update command(s) including at least one analyzer parameter update to the identified point of care analyzer(s) 10.1-10.n which, in turn, can be configured to receive the analyzer update command and to update at least one analyzer parameter according to the corresponding analyzer parameter update. According to embodiment(s) of the disclosed system/method and according to the particular point of care analyzer(s) 10.1-10.n, the updating of an analyzer parameter may become effective immediately, upon the next start-up/restart/docking etc. of the analyzer.

It can be noted, that particulars described with reference to the embodiments of the system as shown on FIG. 1 respectively the method as shown on FIG. 2 apply mutatis mutandis to the embodiments of the system as shown on FIGS. 3 and 5A through 6, respectively the method as shown on the FIGS. 4, 9, 11 and 13, namely embodiments including a dedicated hardware management server 52 and a dedicated remote configuration server 56.

FIG. 5A shows an illustrative diagram of a further embodiment of the disclosed POCT system 1, the remote configuration server 56 further including an authentication and authorization unit 58 and an adapter unit 57.

The authentication and authorization unit 57 can be provided and configured to authenticate and authorize the portable computing device 20 and/or a point of care coordinator (POCC) via the portable computing device 20 (see above a detailed description of the authentication and authorization) with the remote configuration server 56.

The adapter unit 58 can be provided for and configured to adapt the configuration request (received from the portable computing device 20) to the hardware management server interface 53 before transmitting the configuration request to the hardware management server 52. Adapting a configuration request(s) to an interface can refer to enabling communicative data exchange with the particular interface.

Therefore, the term ‘adapt’ or ‘adapting’ as used herein with reference to the adapter unit 58 adapting a configuration request(s) to an interface can include (but is not limited to) one or more of the following:
reformatting/encapsulating the configuration request to a format compatible with the target interface, for example to use standards such as Health Level Seven (HL7) or Fast Healthcare Interoperability Resources (FHIR);
redirecting the configuration request to the respective hardware management server (see FIG. 6 and related paragraphs);
appending authentication and/or authorization data related to the remote configuration server 56 to the configuration request, the authentication and/or authorization data authenticating and authorizing the remote configuration server 56. In other words, the remote configuration server 56 can authenticate the portable computing device 20 based on credentials thereof, but forward the configuration request with its own credentials. This way the remote configuration (via portable computing device(s)) can be transparent to the hardware management server 52;
encrypting the configuration request before transmission;
compressing the configuration request before transmission, such as compressing the request using standard HTTP technology so that network latency can be reduced and performance is improved between the adapter unit 58 and the hardware management server 52.

The modular design shown on FIGS. 5A through 6 allows the system 1 to define multiple adapters based on domain & standard-driven resources so that multiple adapters can be built out of the modules connected to them to fit on different purposes or for being tailored down for simplification purposes. Additionally, the possibility of defining multiple adapters by configuring the modules can allow higher scalability using multiple machines for exposing the final interface combining software and platform as service paradigms.

Further units (not shown on the Figs) of the remote configuration server 56 can include one or more of the following: Common Management, Device Management, Practitioner Management, and/or Organization Management

FIG. 5B shows an embodiment of the disclosed POCT system 1 as communicatively connected to a Laboratory Information System (LIS) and/or Hospital Information system (HIS) 200. It can be readily apparent that the remote configuration services—made available by the disclosed solution including a remote configuration server 56 communicating with the hardware management server 52—may be implemented without affecting an existing installation of a POCT system (with one or more point of care analyzers) and a LIS and/or HIS connected thereto. According to certain embodiments, the existence of the remote configuration server 56—and thus the remote configuration services via a portable computing device 20—can be also agnostic to the LIS and/or HIS.

As shown on FIG. 6 according to further embodiment(s) of the disclosed POCT system 1, the hardware management server 52 can include a first hardware management subserver 52.1 communicatively connected to a first group I of one or more point of care analyzer(s) 10.1-10.n and a second hardware management subserver 52.2 communicatively connected to a second group II of one or more point of care analyzer(s) 10.1-10.n. Multiple hardware management subservers 52.1, 52.2 can be advantageous in POC environments with point of care analyzers of different standards, from different vendors which need to be connected to different standard and/or proprietary hardware management systems. Thus, the first group I of one or more point of care analyzer(s) 10.1-10.n may comprise point of care analyzers of one standard while the second group II of one or more point of care analyzer(s) 10.1-10.n may comprise point of care analyzers of a further standard.

Correspondingly, the first hardware management subserver 52.1 can be configured to transmit the analyzer update command(s) to the one or more point of care analyzer(s) 10.1-10.n of the first group; and the second hardware management subserver 52.2 can be configured to transmit the analyzer update command(s) to the one or more point of care analyzer(s) 10.1-10.n of the second group.

According to embodiments of the disclosed system/method, the first hardware management subserver(s) 52.1 can be configured to expose a first subserver interface 53.1 for communication with the remote configuration server 56 and the second hardware management subserver(s) 52.2 can be configured to expose a second subserver interface 53.2 for communication with the remote configuration server 56, the first and second subserver interfaces 53.1, 53.2 not being necessarily identical. Correspondingly, the adapter unit 58 of the remote configuration server 56 can be configured to adapt the configuration request corresponding to one or more point of care analyzer(s) 10.1-10.n of the first group to the first subserver interface 53.1, in order to accommodate the first subserver interface 53.1 and the adapter unit 58 of the remote configuration server 56 can be configured to adapt the configuration request corresponding to one or more point of care analyzer(s) 10.1-10.n of the second group to the second subserver interface 53.2, in order to accommodate the second subserver interface 53.2. According to further embodiments, the adapter unit 58 can be configured to adapt configuration request(s) to particular hardware management subserver(s) respectively their interface(s) using one or more module(s).

Such embodiments can be particularly advantageous as they can allow the combination of multiple solutions (hardware management servers) by exposing or aggregating their interfaces based on modules of the adapter unit 58. All the modules can be based on plugins so that one can extend the functionality merely by creating new modules.

According to certain embodiments, the first group I of one or more point of care analyzer(s) 10.1-10.n connected to a first hardware management subserver 52.1 respectively the second group II of one or more point of care analyzer(s) 10.1-10.n connected to second hardware management subserver 52.2 can be pre-existing installations of various point of care analyzers, while the remote configuration server 56 can be installed as an add-on to provide remote configuration services across all the different groups of point of care analyzers and across the different hardware management subservers. Also, a third (and further) group(s) of one or more point of care analyzer(s) 10.1-10.n connected to a third (and further) hardware management subserver 52.x may be added to the system without affecting the other group(s) of point of care analyzer(s), the only requirement being that the adapter unit 58 can be further configured to adapt configuration request(s) to the interface of the newly added hardware management subserver.

Therefore, the disclosed system architecture can allow both a horizontal and vertical scaling so that POCT system(s) with large amounts of consumers can maintain optimal performance and reduce or even eliminate downtime upon scaling/extension of the system.

According to certain embodiments, as shown on FIG. 6, in order to connect the different groups of more point of care analyzer(s) 10.1-10.n to the respective hardware management subservers 52.1, 52.2, the POC communication network area 71 may comprise a first POC communication subnetwork 71.1 (shown with dashed lines) to communicatively connect the first group of one or more point of care analyzer(s) 10.1-10.n and the first hardware management subserver(s) 52.1 and a second POC communication subnetwork 71.2 (shown with dotted lines) to communicatively connect the second group of one or more point of care analyzer(s) 10.1-10.n and the second hardware management subserver(s) 52.2.

According to various embodiments, the first POC communication subnetwork 71.1 and the second POC communication subnetwork 71.2 may use the same communication channels. In other words, the different subnetworks 71.1, 71.2 may share the same HW infrastructure, the separation into different subnetworks 71.1, 71.2 being implemented on a software level.

Also shown on FIG. 6, according to certain embodiments of the disclosed system/method, a directory server 59 can be provided to store authentication and authorization data and configured to allow sharing of the authentication and authorization data between the directory server 59 and one or more of the hardware management server 52, the first hardware management subserver 52.1 and the second hardware management subserver 52.2 for authenticating and authorizing the one or more point of care analyzer(s) 10.1-10.n and operator(s) of the one or more point of care analyzer(s) 10.1-10.n.

Also shown on FIG. 6 with dotted-dashed lines, according to certain embodiments of the disclosed system/method, the authentication and authorization unit 57 of the remote configuration server 56 and the directory server 59 can be configured and communicatively connected in order to allow exchange of authentication and authorization data. Thus, the authentication and authorization unit 57 in conjunction with the directory server 59 can allow the connection of multiple different services or software solutions so it can be used for providing single sign-on improving efficiency and user experience of the operator(s).

In the following, different embodiments of the communication network 70 will be described in greater detail. FIG. 8A shows a first embodiment of the communication network 70, wherein the portable computing device 20 can be communicatively connected with the remote configuration server 56 by a remote configuration network area 72, while the one or more point of care analyzer(s) 10.1-10.n are communicatively connected with the hardware management server 52 using a POC communication network area 71. According to an example illustrated, in particular embodiments, the remote configuration network area 72 can be a mobile telecommunication network (such as a wireless mobile Internet service, for example a 3G, 4G or LTE standard) provided by a mobile data carrier service. On the other hand the POC communication network area 71 (of particular embodiments of the communication network 70) can be a separate network, for example a combination of wired and wireless networks, wherein the healthcare facilities (such as different locations/buildings/floors) can be linked by a wired communication network while the individual point of care analyzer(s) 10.1-10.n connect via wireless access points in-between.

As shown on FIG. 8A, according to certain embodiments disclosed, there may be no need for a communication between the portable computing device 20 and the point of care analyzer(s) 10.1-10.n as the (re)configuration thereof can be carried out via the hardware management server 52.

Nevertheless, according to a further embodiment of the communication network 70—as illustrated on FIG. 8B, the portable computing device 20 and the point of care analyzer(s) 10.1-10.n can be communicatively connected by a portable device to analyzer network area 73. In this case, the portable computing device 20 can act as a router/access point for the point of care analyzer(s) 10.1-10.n, “sharing” its network connection therewith. This embodiment can be advantageous in use scenarios when the point of care analyzer(s) 10.1-10.n may not have direct communication with the server 50. This can be the case for example in remote areas where the point of care analyzers 10.1-10.n cannot be connected to a POC communication network area 71 (or not all of them), but the portable computing device 20 does have a network connection via the remote configuration network area 72. In other cases, it may not be economic to provide each point of care analyzer with a network connection. A further scenario—where the portable computing device 20 does have a network connection via the remote configuration network area 72 but not each point of care analyzer—is emergency response, in which case the point of care analyzers 10.1-10.n may need to be deployed “in the field” in a short period of time. The disclosed system/method can be advantageous in such scenarios as well, since even without each point of care analyzer 10.1-10.n having a network connection of their own, they can all be configured easily and consequently from a portable computing device 20, only this portable computing device 20 needing a network connection.

FIG. 8C shows a further embodiment of the communication network 70; where a portable device to analyzer network area 73 can be provided to communicatively connect the portable computing device 20 with a point of care analyzer 10.1-10.n. In this embodiment, the portable computing device 20 may not be directly connected with the remote configuration server 56, but via the portable device to analyzer network area 73, the point of care analyzer(s) 10.1-10.n acting as a router/access point for the portable computing device 20, “sharing” their network connection therewith. This embodiment can be advantageous in POC environments where the point of care analyzers 10.1-10.n can be already communicatively connected with the respective hardware management server(s) 52 but there may be no mobile telecommunication network (no signal) for the portable computing device 20 to connect to the remote configuration server 56. For example there can be POC environments where a mobile telecommunication network is not desired/permitted, the point of care analyzer(s) 10.1-10.n being connected to the respective hardware management server(s) 52 with a wired point of care POC communication network area 71. Nevertheless, a direct communication between the portable computing device 20 and the point of care analyzer(s) 10.1-10.n (for example via Bluetooth or IR) can still be possible/allowable.

FIG. 8D shows a further embodiment of the communication network 70, where in addition to a remote configuration network area 72 and a POC communication network area 71, a portable device to analyzer network area 73 can be also provided. This embodiment can provide both flexibility and redundancy for the communicative connection of the one or more point of care analyzer(s) 10.1-10.n and the portable computing device 20 with the server 50.

It can be pointed out however, that the particular implementation of the communication network 70 (as illustrated on FIGS. 8A to 8D) can be to a certain degree transparent to the POCT system 1 and may not negatively affect the disclosed configuration method(s) as all embodiments of the communication network 70 disclosed herein can be configured to communicatively connect the one or more point of care analyzer(s) 10.1-10.n with the respective hardware management server(s) 52 and the portable computing device 20 with the remote configuration server 56, be it directly or via different network area(s), such as the POC communication network area 71 and/or the remote configuration network area 72 and/or the portable device to analyzer network area 73.

In the following, particular workflows of configuring a POCT system will be described, the implementation of these workflows being simplified and optimized by the hereby described system architecture of the POCT system 1 including a dedicated remote configuration server 56 and a dedicated hardware management server 52 (subservers 52.1-52.x). It will be recognized, that—according to various embodiments of the disclosed system/method—such workflows may be added, edited or removed by altering only the remote configuration server 56 but without affecting the hardware management server 52 (or subservers 52.1-52.x). While describing the particular workflows of configuring a POCT system, the hardware management server 52 (or subservers 52.1-52.x) and the remote configuration server 56 can collectively be referred to as the server 50.

A common task of a POCC can be the relocation of a point of care analyzer(s), necessary due to changed organizational structure or workload fluctuations within the healthcare facilities. The physical relocation of a point of care analyzer(s) usually also can trigger the need to update corresponding settings of the server (hardware management server) and also reconfiguration of one or more parameters of the point of care analyzer(s). For example, when a point of care analyzer(s) is relocated from a children's hospital ward to an adult's ward, certain parameters of the point of care analyzer(s) may need to be updated accordingly to ensure the analyzer provides accurate results. Furthermore, not only parameters of the point of care analyzer(s) but also settings of the server (hardware management server) may need to be updated to reflect the change in authorization of the use of the respective point of care analyzer(s) by personnel in the adult's ward authorized to perform diagnostic procedures on adult's samples.

Using known POCT systems managed by known servers (hardware management server), the POCC may need to catalog all analyzer(s) to be relocated, change their corresponding settings in the server (hardware management server) at his workstation. Also, the POCC may need to physically locate each point of care analyzer(s), change their respective settings to reflect the relocation. This process may not only be time-consuming but also error prone. Therefore the POCC can have no convenient and efficient methods to replace a non-functional point of care analyzer(s) with a backup analyzer(s) using known POCT systems. While this process is feasible in a healthcare facility with a limited number of analyzers, it can be a real challenge when a POCC is responsible for hundreds, if not thousands, of point of care analyzers.

However, none of the known systems can offer convenient workflow(s) to (re)configure point of care analyzers remotely, (re)configuration workflow(s) such as relocation of one or more point of care analyzer(s) or replacement of non-functional point of care analyzer(s) with backup analyzer(s).

According to certain further embodiments of the disclosed POCT system 1 respectively methods for configuration thereof, the configuration command can include a relocation command corresponding to a relocation of one or more point of care analyzer(s) 10.1-10.n, which can be transmitted to the server 50, which can update one or more system parameter(s) in accordance with the relocation of the identified point of care analyzer(s) 10.n-10.n and can retrieve at least one analyzer parameter update in accordance with the relocation of the identified point of care analyzer(s) 10.n-10.n to be transmitted within the analyzer parameter update to the identified point of care analyzer(s) 10.n-10.n.

FIG. 9 shows a use case diagram of an embodiment of the disclosed system/method for configuration of a POCT system 1, illustrating a relocation workflow of a point of care analyzer 10.1-10.n. The relocation can include both a physical and/or logical relocation of one or more point of care analyzer(s) 10.1-10.n. For example a physical relocation can refer to a change of physical location from one healthcare facility/building/building floor, etc. to another. On the other hand, the logical relocation can refer to a change in the reassignment of one or more point of care analyzer(s) 10.1-10.n for example between different hospital wards, emergency departments, intensive care units, primary care setting, medical centers, patient homes, a physician's office, a pharmacy or a sites of an emergency.

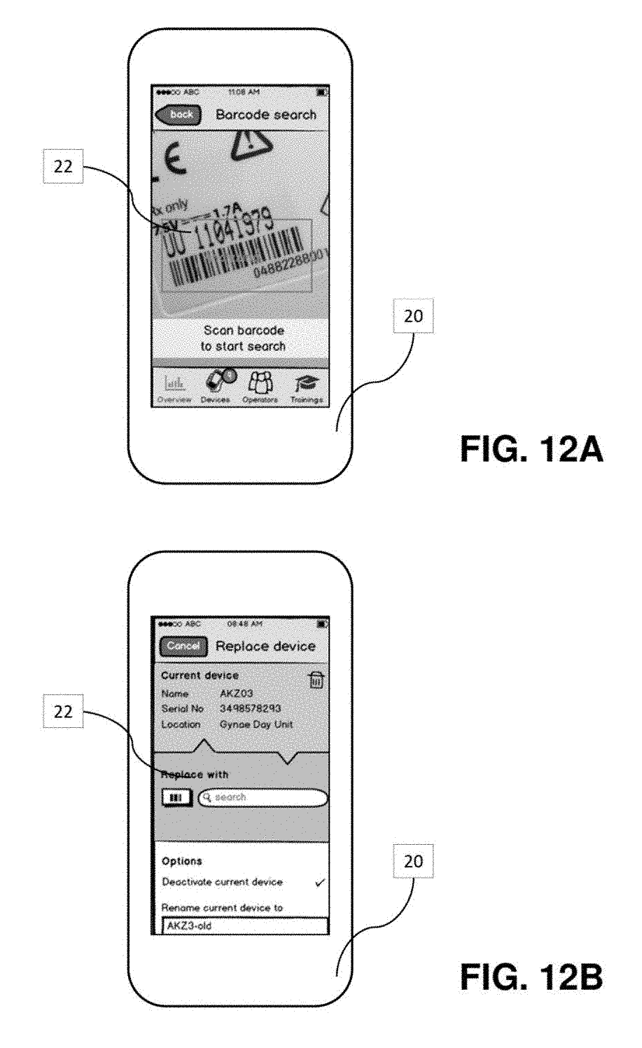

As shown on the use case diagram of FIG. 9 and the screenshots of FIGS. 10A and 10B, the configuration command received by the portable computing device 20 can include a relocation command corresponding to a relocation of one or more point of care analyzer(s) 10.1-10.n. This may be in the form of a selection of a physical and/or logical location from a list, a map or manual input of a location or a combination thereof.

As shown on FIG. 9, according to embodiments of the disclosed system/method directed towards a relocation of point of care analyzer(s) 10.1-10.n, the server 50 can be configured to update one or more system parameter(s) in accordance with the relocation of the identified point of care analyzer(s) 10.1-10.n and to retrieve at least one analyzer parameter in accordance with the relocation of the identified point of care analyzer(s) 10.1-10.n to be transmitted within the analyzer parameter update to the identified point of care analyzer(s) 10.1-10.n. For example, upon receiving a relocation command including as new location a children's hospital ward, the server 50 can retrieve all analyzer parameters such as different reference values (as different values apply when analyzing patient samples of children as compared to adults), new workflow definitions (as different locations may use different workflows), new Quality Control QC parameters, etc. (see definition of system/analyzer parameters above). The analyzer parameters retrieved in this step can be referred to as retrieved analyzer parameters.

As a following step, the server 50 can transmit the retrieved analyzer parameter(s) to the point of care analyzer(s) 10.1-10.n to be relocated (that is the identified point of care analyzer(s) 10.1-10.n by the portable computing device 20).

One further aspect of the maintenance operations of a POCC can be to replace a non-functional point of care analyzer(s) with a backup analyzer(s) and/or to relocate a point of care analyzer(s) as needed, these operations including (re)configuration of the respective point of care analyzer(s) and the server (hardware management server) according to the replacement/relocation of analyzer(s).

Using available POCT system(s) managed by known hardware management servers, the POCC can have to fetch the non-functional point of care analyzer(s) and the backup analyzer(s) and change their allocation on the server at his workstation, updating all the corresponding settings so that the backup analyzer(s) take the place of the non-functional point of care analyzer(s). Thereafter the POCC (or an assistant/nurse etc.) may need to take the (now replaced) point of care analyzer(s) to the location where this is needed. When a POCC is responsible for hundreds of analyzers possibly at different locations, this can be a tedious and time-consuming task, which can be especially problematic in an environment where clinical decision making in the emergency department, intensive care units or primary care setting can be dependent on analysis to be performed by the replacement analyzer.

Alternatively the operator (an assistant/nurse) of the non-functional point of care analyzer(s) can call (or email or notify by other suitable means) the POCC and provide him with the details of the non-functional point of care analyzer(s)—such as its identifier (e.g. barcode) so that the POCC can update all the corresponding settings so that the backup analyzer(s) can take the place of the non-functional point of care analyzer(s). However, this solution can be also time-consuming and error-prone, especially as often it can be only the POCC—who already travelled onsite—the one to determine that the point of care analyzer(s) is non-functional and needs to be replaced. Therefore, the POCC can have no convenient and efficient methods to replace a non-functional point of care analyzer(s) with a backup analyzer(s) using known systems.

According to certain further embodiments of the disclosed POCT system 1 respectively methods for configuration thereof, the configuration command can include a replacement command corresponding to a replacement of a first point of care analyzer 10.1 (e.g. a broken analyzer) with a second point of care analyzer 10.2 (e.g. a replacement analyzer). The replacement command can be transmitted by the portable computing device 20 to the server 50, which can retrieve one or more system parameter(s) corresponding to the first point of care analyzer 10.1, update therewith respective system parameter(s) corresponding to the second point of care analyzer 10.2 and retrieve one or more analyzer parameter(s) corresponding to the first point of care analyzer 10.1 to be transmitted within the analyzer parameter update to the second point of care analyzer 10.2.

FIG. 11 shows a use case diagram of an embodiment of the disclosed system/method for configuration of a POCT system 1, illustrating a replacement workflow of a first point of care analyzer 10.1 with a second point of care analyzer 10.2. The replacement of the first point of care analyzer 10.1 may be required for several reasons, including (but not limited to) replacement of a non-functional (broken) or not-fully-functional (partially broken) analyzer with a backup analyzer with identical or at least similar capability(s) of analyzing one or more patient sample(s). The term ‘non-functional’ or ‘not-fully-functional’ with reference to a point of care analyzer can mean that the particular point of care analyzer may at least at that moment not be capable to perform or not capable to perform at a required quality/speed at least one of its functions, such as the analysis of a patient sample. Thus the term ‘non-functional’ or ‘not-fully-functional’ may not necessarily mean that the point of care analyzer is completely defective/broken. Furthermore, the term ‘non-functional’ or ‘not-fully-functional’ can comprise software- and/or hardware aspects of non-functionality.

Alternatively, an analyzer may be replaced with a different analyzer which is at the time not in use or when the priority of analyzing more patient sample(s) so requires. In a different usage scenario, the replacement workflow of a first point of care analyzer 10.1 with a second point of care analyzer 10.2 can be performed within an update/exchange or maintenance of the POCT system 1.

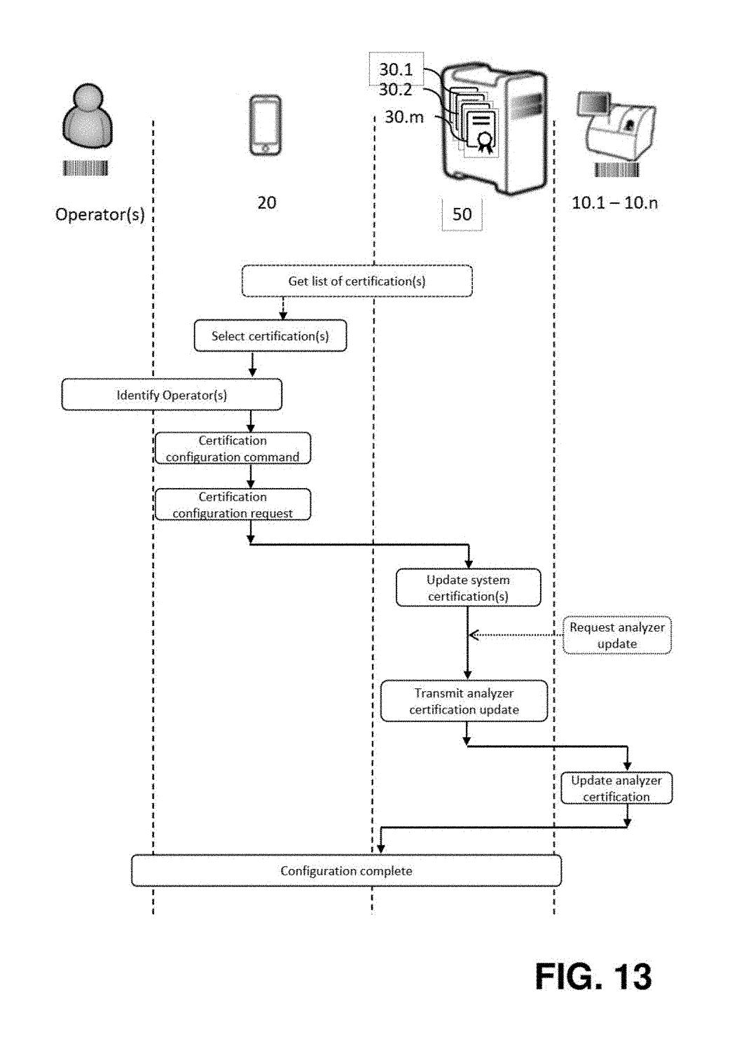

As illustrated on FIG. 11, in a step, the portable computing device 20 can identify a first point of care analyzer 10.1 which may need to be replaced and a second point of care analyzer 10.2 on basis of the corresponding analyzer identifier(s). Thereafter, the device replacement command can be received (as shown on the screenshot of FIG. 12B) corresponding to a replacement of the first point of care analyzer 10.1 with the second point of care analyzer 10.2. According to particular embodiments, the replacement command can be comprised within the configuration command. Alternatively, a separate command may be given by the operator via the user interface 22.

According to alternative embodiments, the replacement command can be received before the second point of care analyzer 10.2 is identified.

After receiving the replacement command and identifying both the first point of care analyzer 10.1 which needs to be replaced and the second point of care analyzer 10.2, the portable computing device 20 can generate the configuration request including the replacement command and the analyzer identifiers corresponding to the first point of care analyzer 10.1 and to the second point of care analyzer 10.2.

As shown on FIG. 11, according to embodiments of the disclosed system/method directed towards an analyzer replacement, the server 50 can be configured to retrieve one or more system parameter(s) corresponding to the first point of care analyzer 10.1 and update therewith respective system parameter(s) corresponding to the second point of care analyzer 10.2. The system parameters retrieved in this step can be referred to as retrieved system parameters. In other words, the system parameters corresponding to the first point of care analyzer 10.1 can be copied/moved to update the system parameter(s) for the second point of care analyzer 10.2. After updating the system parameters, the second point of care analyzer 10.2 can take the place of the first point of care analyzer 10.1 in the POCT system 1. However, in order for the replacement to be fully completed and for the operator to perceive and be able to use the second point of care analyzer 10.2 as it would be the first point of care analyzer 10.1 (at least functionally—in case the two analyzers are not identical in appearance), analyzer parameter(s) corresponding to the first point of care analyzer 10.1 can be retrieved by the server 50 and sent to the second point of care analyzer 10.2 as analyzer parameter update(s). The analyzer parameters retrieved in this step can be referred to as retrieved analyzer parameters.

Since the second point of care analyzer 10.2 can be configured to receive analyzer update command(s) and to update at least one analyzer parameter according to the corresponding analyzer parameter update, the analyzer parameters of the first point of care analyzer 10.1 can be copied/moved to the second point of care analyzer 10.2.

Therefore embodiments of the disclosed system/method directed towards an analyzer replacement can be particularly advantageous as they can provide a simple and efficient workflow solution to replacing a point of care analyzer by a single command given on a portable computing device, wherein the disclosed system/method can take care of the necessary steps (on the server and the involved point of care analyzers) so that thereafter an operator can use the replacement point of care analyzer providing the same functionalities and/or same setup/configuration and/or user rights, etc. (see the non-exhaustive list of system parameter and/or analyzer parameter above) as the POC device which had to be replaced. This can ensure a very positive user experience as service/maintenance and/or analyzer update activities can be made transparent to the operator and downtime of a particular type of point of care analyzer can be eliminated or at least minimized.

According to embodiments of the disclosed system/method, the server 50 can be configured to flag the first point of care analyzer 10.1 as inactive after updating the system parameter(s) corresponding to the second point of care analyzer 10.2. The POCC can then inspect point of care analyzers flagged or have them inspected by a technician. After repair (hardware and/or software), the inactive flag of the respective point of care analyzers can be removed and become available for use again, be it immediately or as a backup analyzer for use as replacement.

A further challenge in the management of POCT can be posed by the configuration management of operator training and of the corresponding training certification(s). Traditionally, the management of operator training/certification(s) can imply that training/certification data has to be entered centrally often even manually in a paper based approach. An obvious consequence of this type of training/certification management can be that the probability of an error or delays is higher. This situation can have a high impact on efficiency and can even create some issues regarding the speed of patient care.

However, the certification management of prior art solutions can be performed centrally. This approach can have several disadvantages, especially in dynamic POC environments with a high number of operators requiring training (in the thousands to tens of thousands across multiple healthcare facilities), a high turnover rate of operators who may be transferred between different departments and operators with diverse educational backgrounds. Thus, in comparison with a more static environment where trainings and examinations can be planned in advance and corresponding certificates can be managed centrally, in a dynamic environment there can be a need to be able to perform the management of operator training/certification/status in an ad-hoc manner and if needed both on-site—namely at or near the particular point of care analyzer, the patient and/or the healthcare staff (the operator)—or off-site—which may be necessary in cases when it is not feasible for the POCC to relocate (for time/distance/resource constraints).

According to certain further embodiments disclosed, the configuration of the POCT system 1, in order to manage operator certifications, can be performed as follows:
one or more system certification(s) 30.1-30.m, each corresponding to one or more point of care analyzer(s) 10.1-10.n can be stored on the server 50;
operators of the POCT system 1 can be provided with corresponding operator identifier(s);
one or more certification(s) 30.1-30.m can be selected via the user interface 22 of the portable computing device 20;
one or more operator(s) can be identified using the operator identifier(s);
a certification configuration command can be given via the user interface 22 of the portable computing device 20;
a certification configuration request can be generated by the portable computing device 20, the certification configuration request comprising the one or more operator identifier(s) corresponding to the identified operator(s) and data identifying the selected system certification(s) 30.1-30.m;
the server can receive the configuration request;
the server 50 can update the selected system certification(s) 30.1-30.m according to the certification configuration request;
the server 50 can transmit an analyzer certification update for each of the selected system certification(s) 30.1-30.m to the corresponding point of care analyzer(s) 10.1-10.n;
the one or more point of care analyzer(s) 10.1-10.n can receive the analyzer certification update;
the one or more point of care analyzer(s) 10.1-10.n can update their respective analyzer certification according to the analyzer certification update.

Such embodiments of the disclosed system/method can be particularly advantageous for allowing a decentralized control of analyzer certifications and permitting that a POCC easily adds/deletes/updates operators (their status) to the system. Status update(s) can be related to the completion of training/successful examination or an operator access update. This way such management steps can be done immediately via a portable computing device in an efficient and secure manner.

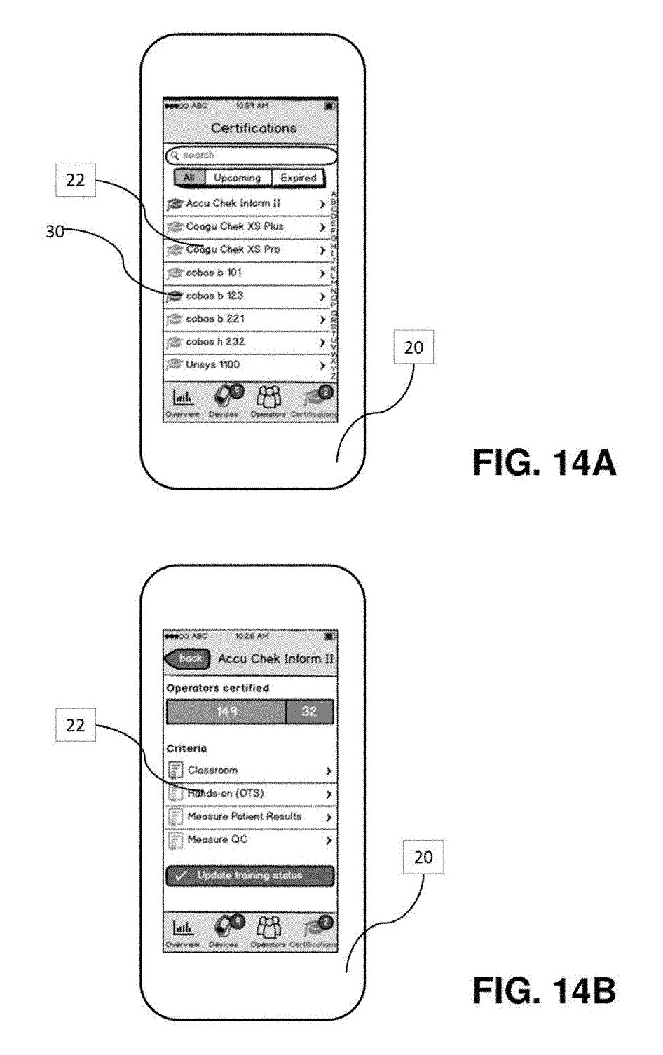

FIG. 13 shows a use case diagram of further embodiments of the disclosed system/method for configuration of a POCT system, illustrating a certification configuration corresponding to one or more operator(s) and one or more certification(s).

According to embodiments of the disclosed system/method, the server 50 can be further configured for storing one or more system certification(s) 30.1-30.m, each corresponding to one or more point of care analyzer(s) 10.1-10.n. As shown on FIG. 10A, according to particular embodiments, the server 50 can be configured to store a system certification 30.1-30.m for each type/class of point of care analyzers. For this, analyzers of the same type/class (with similar functionality of analyzing patient sample(s)) can be grouped and a system certification 30.1-30.m can be stored for each type/class, each system certification 30.1-30.m storing the operator identifier(s) of each operator certified to operate one or more point of care analyzer(s) 10.1-10.n of the type/class. Alternatively, the server 50 can be configured to store a system certification 30.1-30.m for each of the one or more point of care analyzer(s) 10.1-10.n.

In a step (of certain embodiments—the step being shown with dotted lines) the portable computing device 20 can be configured to request the one or more system certifications 30.1-30.m and/or a list of the one or more system certifications 30.1-30.m from the server 50, while the server 50 can be configured to transmit the one or more system certifications 30.1-30.m and/or a list of the one or more system certifications 30.1-30.m to the portable computing device 20.

Thereafter, one or more of the system certifications 30.1-30.m can be selected via the user interface 22 of the portable computing device 20 as shown on the screenshot of FIG. 10A, these are referred to as the selected system certifications 30.1-30.m.

For identifying operator(s) of the one or more point of care analyzer(s) 10.1-10.n of the POCT system 1, operator(s) can be provided with operator identifier(s), each operator identifier uniquely identifying the respective operators. Correspondingly, the portable computing device 20 can be configured to identify one or more operator(s) of the POCT system 1 using one or more operator identifier(s). The one or more operator(s) identified by the portable computing device 20 can be referred to as the identified operator(s).

According to embodiments of the disclosed system/method, the one or more operator identifier(s) can be identifier tags, such as a barcode and/or an RFID tag and/or an alphanumeric identifier. Correspondingly, the portable computing device 20 can include an identifier reader such as a barcode reader and/or an RFID reader to read the identifier tag and/or input (such as a keyboard or input field on a screen) for inputting an alphanumeric identifier of the one or more operator identifier(s). In addition or alternatively, a camera device may be provided to identify the operators based on the operator identifier(s). In addition or alternatively, biometric identification of the operator(s) may be used.

After one or more operator(s) have been identified and one or more certifications 30.1-30.m has been selected, a certification configuration command can be received via the user interface 22 of the portable computing device 20. According to embodiments of the user interface 22, the certification configuration command may be a push of a button (physical or screen button) a voice command, a selection in a menu, etc. The certification configuration command may be any form of input from an operator to initiate a configuration workflow (process).

Initiated by the certification configuration command, the portable computing device 20 can generate a certification configuration request comprising the one or more operator identifier(s) corresponding to the identified operator(s) and data identifying the selected system certification(s) 30.1-30.m.

According to embodiments of the disclosed system/method, the certification configuration request transmitted by the portable computing device 20 to the server 50 includes one or more of the following: addition of an operator status corresponding to the identified operator(s) and/or removal of an operator status corresponding to the identified operator(s) and/or update of an operator status corresponding to the identified operator(s).

After generating it (optionally after confirmation from the operator), the portable computing device 20 can transmit the certification configuration request to the server 50. Therefore, the certification configuration request can be described as a sort of translation of the certification configuration command from the operator into a request signal to the server 50.

After receiving the certification configuration request—via the communication network 70—the server 50 can update the selected system certification(s) 30.1-30.m according to the certification configuration request and can transmit an analyzer certification update for each of the selected system certification(s) 30.1-30.m to the corresponding point of care analyzer(s) 10.1-10.n.

The above steps by the server 50 will now be described in greater detail. On one hand, the server 50 can be configured to update the selected system certification(s) 30.1-30.m within the server 50 according to the certification configuration request. On the other hand, in order to ensure that the certification configuration command (as a complete workflow) is implemented not only on the server 50 but across the POCT system 1, the server 50 can be configured to transmit an analyzer update for each of the selected system certification(s) 30.1-30.m to the corresponding point of care analyzer(s) 10.1-10.n.

According to embodiments of the disclosed system/method, the analyzer certification update transmitted by the server 50 to the corresponding point of care analyzer(s) 10.1-10.n can include one or more of the following: one or more operator identifier(s) corresponding to the identified operator(s) to be granted access the respective point of care analyzer 10.1-10.n and/or one or more operator identifier(s) corresponding to the identified operator(s) to be denied access to the respective point of care analyzer 10.1-10.n and/or one or more operator identifier(s) corresponding to the identified operator(s) to be granted limited access to the respective point of care analyzer 10.1-10.n.

According to embodiments of the disclosed system/method, the server 50 transmitting an analyzer certification update for each of the selected system certification(s) 30.1-30.m to the corresponding point of care analyzer(s) 10.1-10.n, the one or more point of care analyzer(s) 10.1-10.n receiving the analyzer certification update, and the one or more point of care analyzer(s) 10.1-10.n updating their respective analyzer certification according to the analyzer certification update can be initiated by one or more point of care analyzer(s) 10.1-10.n requesting an update of the corresponding system certification(s) 30.1-30.m. Correspondingly, the one or more point of care analyzer(s) 10.1-10.n can be configured to request analyzer certification update(s) on occurrence(s) of certain event(s) and/or at regular intervals. According to embodiments of the disclosed system/method, the one or more point of care analyzer(s) 10.1-10.n can be configured to request an analyzer certification update upon a login by an operator and/or upon startup and/or upon shutdown and/or upon docking (into a docking station) of the respective point of care analyzer 10.1-10.n. This can be shown on the use case diagrams with dotted lines.

To summarize, the analyzer certification can update(s)—according to embodiment(s) of the disclosed system/method—the analyzer certification update(s) may be implemented using “server push” technology (that is server initiated) and/or a “client pull” technology (client—in this case point of care analyzer initiated). The server push implementation of analyzer certification update(s) can be advantageous for point of care analyzers 10.1-10.n which can be continuously communicating with the server, while the client pull implementation of analyzer certification update(s) can be advantageous for point of care analyzers 10.1-10.n which communicate with the server only on an event basis, such as periodically and/or upon a login by an operator and/or upon startup and/or upon shutdown of the respective point of care analyzer 10.1-10.n. Also, according to embodiments of the disclosed system/method, the point of care analyzers 10.1-10.n can request an analyzer certification update from the server 50 upon expiry of a validity of the respective system certification(s) 30.1-30.m.

According to particular embodiments of the disclosed system/method, as shown on the screenshot of FIG. 10B, receiving the certification configuration command via the user interface 22 of the portable computing device 20 can include a selection of one or more certification criteria via the user interface 22. Certification criteria can include (but is not limited to) one or more of the following: Classroom training, Hands-on training, Training on patient sample analysis, and/or Training on calibration and/or quality control.

In embodiments of the disclosed system/method according to which one or more certification criteria corresponding to one or more system certification(s) 30.1-30.m are defined, the one or more point of care analyzer(s) 10.1-10.n can be configured to control access of the identified operator(s) to the respective point of care analyzer 10.1-10.n according to the one or more certification criteria of the selected system certification(s) 30.1-30.m. Such control of access can include allowing an operator to use only certain functions of the point of care analyzer 10.1-10.n. For example, an operator who has the certification criterion “Training on patient sample measurement” but not the certification criterion “Training on calibration and/or quality control” may only perform patient sample analysis with the respective point of care analyzer(s) 10.1-10.n but not calibration and quality control.

According to embodiments of the disclosed system/method, the server 50 can be further configured to mark a list of authorized operators for each of the point of care analyzer(s) 10.1-10.n corresponding to the selected system certification(s) 30.1-30.m as invalid and/or update a list of authorized operators for each of the point of care analyzer(s) 10.1-10.n corresponding to the selected system certification(s) 30.1-30.m according to the analyzer certification update. In such embodiment(s) the analyzer certification update can include a list of authorized operators for the respective point of care analyzer(s) 10.1-10.n.

According to embodiments of the disclosed system/method, the point of care analyzer(s) 10.1-10.n can be configured to perform one or more of the following: blood glucose testing, coagulation testing, blood gas or electrolytes analysis, urinalysis, cardiac markers analysis, hemoglobin diagnostics, infectious disease testing, cholesterol screening, and/or nucleic acid testing (NAT).

According to embodiments of the disclosed system/method, the portable computing device 20 can be one of the following: a mobile phone, in particular a smartphone, a tablet computer, a laptop computer, or a dedicated PDA device.

According to embodiments of the disclosed system/method, the server 50 can be configured to retrieve analytical data from the one or more point of care analyzer(s) 10.1-10.n such as data representing the measurement of patient health parameter(s) and update program data of the one or more point of care analyzer(s) 10.1-10.n such as a software update.

It will be understood that not all steps of the methods herein disclosed are necessarily carried out in the listed/described order. In particular, a configuration command may be received by the portable computing device before identifying point of care analyzer(s); operators may be identified before selecting a certification; analyzer update command(s) may be transmitted by the server to the point of care analyzer(s) before updating system parameter(s) or a first point of care analyzer may be flagged as inactive before the second (replacement) point of care analyzer is updated with the analyzer parameters of the first point of care analyzer.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A point of care testing (POCT) system for analyzing biological samples, the POCT system comprising:
one or more point of care analyzer(s) for analyzing one or more patient sample(s), wherein the one or more point of care analyzer(s) are configured to receive the analyzer update command and to update at least one analyzer parameter according to the respective analyzer parameter update;
a portable computing device;
a hardware management server for storing system parameter(s) corresponding to the one or more point of care analyzer(s), wherein the hardware management server is configured to receive and process the configuration request in order to: update at least one system parameter according to the configuration request; and transmit an analyzer update command comprising at least one analyzer parameter update to one or more point of care analyzer(s) according to the configuration request;
a remote configuration server, wherein the remote configuration server is configured to: authenticate and authorize the portable computing device, receive a configuration request from the portable computing device, and transmit the configuration request to the hardware management server; and
a communication network configured to communicatively connect:
the point of care analyzer(s) with the hardware management server,
the portable computing device with the remote configuration server, and
the remote configuration server with the hardware management server.

2. The POCT system for analyzing biological samples according to claim 1, wherein the hardware management server is configured to expose a hardware management server interface for communication with the remote configuration server and wherein the remote configuration server comprises an authentication and authorization unit to authenticate and authorize the portable computing device and/or a point of care coordinator (POCC) via the portable computing device with the remote configuration server and an adapter unit configured to adapt the configuration request to the hardware management server interface.

3. The POCT system for analyzing biological samples according to claim 1, wherein the hardware management server is further configured to perform one or more of the following: retrieving analytical data from the point of care analyzer(s), updating program data of the point of care analyzer(s), and transferring authentication data update(s) and/or certification update data of the point of care analyzer(s).

4. The POCT system for analyzing biological samples according to claim 1, wherein the hardware management server is communicatively connected to one or more of a Laboratory Information System (LIS) and a Hospital Information system (HIS).

5. The POCT system for analyzing biological samples according to claim 1, wherein the communication network comprises a point of care (POC) communication network area for communicatively connecting the point of care analyzer(s) with the hardware management server and a remote configuration network area for communicatively connecting the portable computing device with the remote configuration server.

6. The POCT system for analyzing biological samples according to claim 1, further comprises,
a first hardware management subserver communicatively connected to a first group of one or more point of care analyzer(s), wherein the first hardware management subserver is configured to transmit the analyzer update command(s) to the one or more point of care analyzer(s) of the first group; and
a second hardware management subserver communicatively connected to a second group of one or more point of care analyzer(s), wherein the second hardware management subserver is configured to transmit the analyzer update command(s) to the one or more point of care analyzer(s) of the second group.

7. The POCT system for analyzing biological samples according to claims 6, wherein the first hardware management subserver(s) is configured to expose a first subserver interface for communication with the remote configuration server, the second hardware management subserver(s) is configured to expose a second subserver interface for communication with the remote configuration server, the adapter unit of the remote configuration server is configured to adapt the configuration request corresponding to one or more point of care analyzer(s) of the first group, in order to accommodate the first subserver interface, and the adapter unit of the remote configuration server is configured to adapt the configuration request corresponding to one or more point of care analyzer(s) of the second group, in order to accommodate the second subserver interface.

8. The POCT system for analyzing biological samples according to claim 2, further comprising,
a directory server configured to store authentication and authorization data and configured to allow sharing of the authentication and authorization data between the directory server and one or more of the hardware management server, the first hardware management subserver, the second hardware management subserver for authenticating and authorizing the one or more point of care analyzer(s) and operator(s) of the one or more point of care analyzer(s).

9. The POCT system for analyzing biological samples according to claim 8, wherein the authentication and authorization unit of the remote configuration server and the directory server are configured and communicatively connected in order to allow exchange of authentication and authorization data.

10. A method for configuration of a point of care testing (POCT) system for analyzing biological samples, the method comprising:
providing one or more point of care analyzer(s) for analyzing one or more patient sample(s);
providing a portable computing device;
providing a hardware management server configured for storing system parameter(s) corresponding to the one or more point of care analyzer(s);
providing a remote configuration server;
communicatively connecting the point of care analyzer(s) with the hardware management server, the portable computing device with the remote configuration server, and the remote configuration server with the hardware management server via a communication network;
authenticating and authorizing the portable computing device by the remote configuration server;
receiving a configuration request by the remote configuration server from the portable computing device;
transmitting the configuration request by the remote configuration server to the hardware management server;
receiving and processing of the configuration request by the server, the processing comprising updating at least one system parameter according to the configuration request and transmitting an analyzer update command comprising at least one analyzer parameter update to one or more point of care analyzer(s) according to the configuration request; and
receiving the analyzer update command and updating at least one analyzer parameter according to the respective analyzer parameter update by the one or more point of care analyzer(s).

11. The method for configuration of a POCT system for analyzing biological samples according to claim 10, further comprising,
exposing a hardware management server interface for communication with the remote configuration server by the hardware management server;
providing an authentication and authorization unit of the remote configuration server;
authenticating and authorizing the portable computing device and/or a point of care coordinator (POCC) via the portable computing device with the remote configuration server by the authentication and authorization unit;
providing an adapter unit of the remote configuration server; and
adapting of the configuration request to the hardware management server interface by the adapter unit.

12. The method for configuration of a POCT system for analyzing biological samples according to claim 11, wherein adapting the configuration request received from the portable computing device comprising formatting and/or reformatting the configuration request to a format compatible with the hardware management server interface and adding authentication and/or authorization data to the configuration request to authenticate and authorize the remote configuration server with the hardware management server.

13. The method for configuration of a POCT system for analyzing biological samples according to claim 10, further comprises,
communicatively connecting a first group of one or more point of care analyzer(s) with a first hardware management subserver(s);
communicatively connecting a second group of one or more point of care analyzer(s) with a second hardware management subserver(s);
transmitting the analyzer update command(s) by the first hardware management subserver to the one or more point of care analyzer(s) of the first group; and
transmitting the analyzer update command(s) by the second hardware management subserver to the one or more point of care analyzer(s) of the second group.

14. The method for configuration of a POCT system for analyzing biological samples according to claim 13, further comprises,
exposing by the first hardware management subserver a first subserver interface for communication with the remote configuration server;
exposing by the second hardware management subserver a second subserver interface for communication with the remote configuration server;
adapting the configuration request corresponding by the adapter unit to one or more point of care analyzer(s) to the first subserver interface; and
adapting the configuration request corresponding by the adapter unit to one or more point of care analyzer(s) of the second group to the second subserver interface.

15. The method for configuration of a POCT system for analyzing biological samples according to claim 10, further comprises,
providing a directory server;
storing authentication and authorization data on the directory server; and
sharing of authentication and authorization data between the directory server and the hardware management server and/or the first hardware management subserver and/or the second hardware management subserver for authenticating and authorizing the one or more point of care analyzer(s) and/or operator(s) of the one or more point of care analyzer(s).
